(19) United States
(12) Patent Application Publication
(10) Pub. No.: US 2023/0063506 A1
(43) Pub. Date: Mar. 2, 2023

(54) SMALL RNA DISEASE CLASSIFIERS

(71) Applicant: SRNALYTICS, INC., Needham, MA (US)

(72) Inventors: David W. SALZMAN, Natick, MA (US); Alan P. SALZMAN, Natick, MA (US); Neal C. FOSTER, Natick, MA (US); Nathan S. RAY, Natick, MA (US); Terran MELCONIAN, Natick, MA (US)

(21) Appl. No.: 17/794,047

(22) PCT Filed: Jan. 22, 2021
(86) PCT No.: PCT/US2021/014755

§ 371 Date: Jul. 20, 2022

Related U.S. Application Data
(60) Provisional application No. 62/964,412, filed on Jan. 22, 2020.

(51) Int. Cl. C12Q 1/6886; C12Q 1/6869; G16B 30/10; G06N 20/00

(52) U.S. Cl. CPC C12Q 1/6886; C12Q 1/6869; G16B 30/10; G06N 20/00; C12Q 2600/178

(57) ABSTRACT

The present disclosure provides methods for constructing disease classifiers for evaluating subjects for one or more distinct biological conditions or one or more disease subtypes. The present invention involves identifying candidate small RNA (sRNA) sequences from sequence data of a discovery sample set. The presence or abundance of the candidate sRNA sequences (each taken individually) across the discovery sample set is predictive of a biological condition of interest (e.g., over other distinct biological conditions or non-disease controls), and these candidate sRNA sequences are further filtered or selected in accordance with embodiments of the present disclosure. Machine learning techniques are then applied to build and train disease classifiers, including multi-disease classifiers. The trained classifiers can be used to classify new samples, for example, to evaluate patients for disease.

### PRIORITY

This Application claims the benefit of, and priority to, U.S. Provisional Application No. 62/964,412 filed Jan. 22, 2020, which is hereby incorporated by reference in its entirety.

### BACKGROUND

Diagnostic and therapeutic advances in complex diseases have found only limited success. In contrast to Mendelian disorders, complex disease is often defined as a phenotype that is not caused by a single gene mutation. Complex diseases can be caused by numerous genetic events, which may vary across afflicted individuals, and may include a significant contribution from environmental factors. Conventional approaches to the study of complex disease have identified patients with similar phenotypes and have attempted to identify common causative genetic events for the phenotype using association studies. These approaches operate at the DNA level, for example, by identifying gene mutations such as single nucleotide polymorphisms (SNPs) that are associated with the phenotype. This classical approach has found only limited success, with many expensive drug trials failing to show efficacy, in part because the underlying disease remains poorly characterized or understood, or remains heterogeneous with the established or recognized disease characterizations. See Jameson L J et al., Precision Medicine—Personalized, Problematic and Promising, NEJM 372:2229-2234 (2015); Lyman G H, at al., Biomarker Tests for Molecularly Targeted Therapies—Laying the Foundation and Fulfilling the Dream, J. Clin. Oncol. 34(17):2061-2066 (2016).

New approaches are needed for classifying disease, including approaches for subtyping complex disease. Accurate molecular approaches for classifying or subtyping complex disease could lead to enormous breakthroughs in diagnosis and therapy, and could lead the next generation of patient care. The present invention meets these and other objectives.

### SUMMARY

The present disclosure provides methods for constructing disease classifiers for evaluating subjects for one or more distinct biological conditions or one or more disease subtypes. The present invention involves identifying candidate small RNA (sRNA) sequences from sequence data of a discovery sample set. The presence or abundance of the candidate sRNA sequences (each taken individually) across the discovery sample set is predictive of a biological condition of interest (e.g., over other distinct biological conditions or non-disease controls), or is predictive of disease progression or response to treatment, and these candidate sRNA sequences are further filtered or selected in accordance with embodiments of the present disclosure. Machine learning techniques are then applied to build and train classifiers, including disease classifiers, multi-class disease classifiers, and classifiers of a distinct disease state or condition. The trained classifiers can be used to classify new samples, for example, to evaluate patients for disease or predict groups of diseased patients who will respond to a therapeutic treatment modality.

In some embodiments, the disease classifier is a multi-class predictor. For example, the multi-class predictor may distinguish biological conditions of interest, such as conditions that can manifest with similar clinical symptoms (e.g. dementia, movement disorder, etc.) and/or which have similar pathologic annotation (e.g. disease stage, fibrosis, inflammation, etc.). The candidate sRNA sequences, and particularly their binary profiles (presence or absence) or abundance level profiles across a discovery set, are used to construct the disease classifier using various machine learning models, as described more fully herein. The disease classifier can be used to screen or evaluate subjects for the presence of one or more disease conditions using molecular detection assays, or, in other embodiments, using sRNA sequencing.

In some embodiments, the presence or absence, or abundance, of the candidate sRNA sequences in the discovery set is used to identify or classify disease subtypes. Disease subtypes include diseases that are phenotypically similar, but which may result from disparate dysregulation of biological pathways, or disparate sRNA biogenesis. The disparate subtypes may respond differently to therapeutic interventions. Further, by mapping the predictive sRNA sequences to target genes and their biological pathways, distinct druggable targets and therapeutic regimens for the disease subtypes can be elucidated. Disease subtype classifiers find use in personalized medicine applications, to match patients with the appropriate therapeutic regimen. Disease subtype classifiers further find use in clinical trial design, to tailor patient recruitment to the mechanism of action of the investigational drug.

In various embodiments, the invention provides a method for generating a classifier to evaluate a subject for one or more biological conditions. The method comprises providing sRNA sequence data comprising a compilation of the distinct sRNA sequences that are present across a set of discovery samples, and selecting candidate sRNA sequences whose presence or absence, or abundance (e.g., expression level), is correlative with the presence, absence, stage, or other feature(s) of a biological condition(s) of interest. These distinct sRNA variations (e.g., isomiRs) are not consolidated based on a reference sequence or genetic locus, and thus differs from the conventional approach for analyzing miRNA. The set of discovery samples will generally comprise samples representing the presence or absence of one or more biological conditions of interest, and may further comprise non-diseased controls. A classifier is then trained using various machine learning models, e.g., using the presence or absence, or in some embodiments the abundance, of the candidate sRNA sequences across a training set along with sample metadata comprising clinical phenotypes or pathological labels. The classifier in accordance with this aspect will comprise sRNA features for evaluating a subject's sample for the presence and/or absence of the biological condition(s).

In various embodiments, the discovery set samples are labelled as being positive or negative for the one or more biological conditions of interest. In such embodiments, the invention involves identifying sRNA panels and features for classifying samples using supervised machine learning models. In these embodiments, the invention provides classifiers for accurately classifying biological conditions that may present with similar symptoms or pathologies, including early stages of disease. Examples include CNS disorders that present with dementia or tremors, and disorders that present with gastrointestinal inflammation, among many others. Other disease phenotypes that may be shared across several distinct disease conditions are provided elsewhere herein.

In still other embodiments, the discovery set samples represent samples of a complex disease and non-diseased controls. For example, the complex disease may involve one or more disease subtypes that are not labeled in the discovery set. In some embodiments, the method described herein identifies, potentially for the first time, such disease subtypes. In these embodiments, the invention identifies sRNA features for classifying samples for the presence or absence of such disease subtype(s) using unsupervised or semi-supervised machine learning. Thus, even where surrogate markers are unavailable for labelling samples or where pathologist evaluations are insufficient to distinguish distinct disease subtypes, the presence or absence, or relative abundance, of candidate sRNA sequences in accordance with the invention provides surprisingly effective means to classify samples. In some embodiments, the invention as described herein is used to identify and classify these disease subtypes from discovery sample sets that are otherwise considered pathologically similar.

In order to improve machine learning, the distinct sRNA sequences, which may be around the order of 100 million distinct sequences in a training set, are filtered to several thousand candidate sRNAs using preselection criteria. The candidate sRNA sequences can be selected based on the degree to which their presence, absence, or abundance correlates to the presence or absence of a biological condition of interest. In some embodiments, at least one candidate sRNA sequence is only present in discovery samples (e.g. a training set) that are positive for a biological condition of interest, and absent in all other discovery samples. In some embodiments, at least one candidate sRNA sequence is only present in discovery samples (e.g. a training set) that are negative for a biological condition of interest (e.g., non-disease controls or other biological condition class), and absent in all samples labeled as positive for the biological condition of interest. In various embodiments, candidate sRNA sequences are selected that individually predict, by their presence or absence, for a biological condition of interest in a training set. That is, candidate sRNAs include sequences selected individually for their predictive power in determining the presence or absence of at least one biological condition, against other biological conditions represented in a training set and/or non-disease controls. In some embodiments, candidate sRNA sequences are selected from the sequence data, based on the degree to which their abundance (e.g., over-abundance or under-abundance) correlates to the presence or absence of a biological condition of interest.

In some embodiments, the set of discovery samples are further labeled for stage, grade, or other characteristic(s) of one or more biological condition(s) of interest. In these embodiments, candidate sRNAs may be selected whose read counts correlate with disease activity, such as, for example, disease stage or grade. For example, as disease stage or grade progresses, candidate sRNA sequences can be selected that show higher or lower read counts. That is, average read counts increase or decrease in later stages of the disease, or with higher disease activity. Alternatively, as the disease stage decreases (e.g. in a treatment group), candidate sRNA sequences can be selected that show lower or higher read counts in treated subjects.

In various embodiments, sRNA families (for example, miRNAs having the same seed sequence) are identified that have increased sequence diversity in a biological condition of interest. sRNA isoforms within these sRNA families are selected as candidate sRNA sequences for classification. For example, in some embodiments, sRNA families can be identified in which sequence variation increases in a disease condition and/or increases with severity of a disease condition, and/or which variation may normalize or be ameliorated in response to a therapeutic regimen. In some embodiments, sRNA preselection for machine learning weights toward selection of isomiRs having the same seed sequence, or weights toward other sRNA properties such as isomiRs having variations associated with presence in exosomes (e.g., the presence of a 3′ non-templated nucleotide).

After the sRNA features are selected, a machine learning classifier can be trained, using one or more machine learning approaches. In some embodiments, the classifier is configured to classify samples of a test set based on the presence or absence, or the abundance, of a panel of candidate sRNA. The size of the panel may depend on the number of classes involved. For example, the panel may contain from 1 to about 50,000 sRNA sequence. In some embodiments, the panel contains from about 4 to about 200 sRNA sequences. The maximum size of the panel can be selected in some embodiments (e.g., about 100 sRNAs). In some embodiments, the classifier is based on, for example, a support vector machine algorithm, a decision tree algorithm, an unsupervised clustering algorithm, a supervised clustering algorithm, a logistic regression algorithm, a mixture model, a hidden Markov model, or a neural network algorithm.

The trained machine learning classifier can be used for evaluation of independent subjects for the disease conditions or disease subtypes (biological conditions), by detecting the presence or absence, or abundance, in a biological sample from the subject, of sRNA markers in the panel, and applying the classifier. The biological sample can be assigned to more than one class, with a corresponding probability, or another measure, computed with respect to each class being tested. In some cases, only the assignments with associated probability values above a certain threshold can be provided by the classifier. Furthermore, in some embodiments, a treatment recommendation or regimen can be generated based on the results of the classification of the subject's biological sample.

In other aspects, the invention provides a method for evaluating a subject for one or more disease conditions, or disease subtype. In various embodiments, the method comprises providing a biological sample of the subject, and determining the presence or absence or abundance of sRNAs in an sRNA panel. This sRNA profile is then used to classify the condition of the subject among one or more disease conditions or disease subtypes, using a disease classifier prepared according to this disclosure. Where the patient's condition or disease subtype is identified, the patient can be matched (i.e., administered) to the appropriate therapeutic regimen for the disease condition, and/or included or excluded from a clinical trial. For example, in some embodiments, the patient is administered a therapy that targets a dysregulated or aberrant pathway, and which corresponds to a pathway targeted by one or more sRNAs (e.g., miRNAs) in the panel used for cluster analysis.

In various embodiments, the presence or absence, or abundance, of sRNAs in the subject's sample is determined by a molecular diagnostic assay, such as a quantitative PCR assay. For example, detection of the sRNA sequences is migrated to one of various detection platforms (e.g., other than RNA sequencing), which can employ reverse-transcription, amplification, and/or hybridization of a probe, including quantitative or qualitative PCR, e.g., RealTime PCR. PCR detection formats can employ stem-loop primer(s) for RT-PCR in some embodiments, and optionally in connection with fluorescently-labeled probes.

In still other embodiments, sRNAs that are present in the subject's sample are determined or quantified by sRNA sequencing and adaptor trimming as described elsewhere herein. sRNA sequencing can involve target capture (target-enriched sequencing) as known in the art.

Other aspects and embodiments of the invention will be apparent from the following detailed description.

### DESCRIPTION OF THE FIGURES

FIG. 1 is a flowchart illustrating a method of generating a classifier in accordance with some embodiments.

FIG. 2 is a flowchart illustrating a method of applying the classifier generated using the method of FIG. 1, accordance with some embodiments.

FIGS. 3A-3D depict ROC/AUC curves for various IBD classes and controls, illustrating a highly accurate multi-class disease prediction: Control (FIG. 3A), Crohn's disease (FIG. 3B), Ulcerative colitis (FIG. 3C), and Diverticular disease (FIG. 3D).

FIG. 4 depicts a heat map showing the proportion of accurate multi-class disease predictions against their true reference identities. Classes are Crohn's Disease, Control (CTR), Diverticular Disease, and Ulcerative Colitis.

FIG. 5 illustrates an example of normalization using spike-in small RNA.

FIG. 6A and FIG. 6B illustrate a method for subtyping a complex disease using a combination of supervised and unsupervised machine learning (FIG. 6A). Steps for unsupervised machine learning according to some embodiments are shown diagrammatically in FIG. 6B.

FIG. 7 shows enhanced classifier performance when miRNA variants with common seed regions are aggregated during preselection of sRNAs.

### DETAILED DESCRIPTION OF EMBODIMENTS

The present disclosure provides methods for constructing disease classifiers for evaluating subjects for one or more distinct biological conditions or one or more disease subtypes (sometimes collectively referred to as “biological conditions” or “disease conditions”). The present invention involves identifying candidate small RNA (sRNA) sequences from sequence data of a discovery sample set. The presence or abundance of the candidate sRNA sequences (each taken individually) across the discovery sample set (or training set) is predictive of a biological condition of interest (e.g., over other distinct biological conditions or non-disease controls), and these candidate sRNA sequences are further filtered or selected in accordance with embodiments of the present disclosure. Machine learning techniques are then applied to build and train disease classifiers, including multi-disease classifiers and disease sub-type classifiers. The trained classifiers can be used to classify new samples, for example, to evaluate patients for disease.

In some embodiments, the disease classifier is a multi-class predictor. For example, the multi-class predictor may distinguish biological conditions of interest, such as conditions that typically manifest or present with similar clinical symptoms (e.g., dementia, movement disorder, etc.). The candidate sRNA sequences, and particularly their binary profiles (presence or absence) or expression level profiles across a discovery set, are used to construct the disease classifier using various machine learning models, as described more fully herein. The disease classifier can be used to evaluate subjects for the presence of one or more disease conditions using molecular detection assays, or, in other embodiments, using sRNA sequencing.

In some embodiments, an sRNA panel is used to identify or classify disease subtypes. Disease subtypes include diseases that are phenotypically similar, but which may result from different aberrant or dysregulated biological pathways, or disparate sRNA biogenesis. The disparate subtypes may respond differently to therapeutic interventions. Further, by mapping the predictive sRNA sequences to target genes and their biological pathways, distinct druggable targets and therapeutic regimens for the disease subtypes can be elucidated. Disease subtype classifiers find use in personalized medicine applications, to match patients with the appropriate therapeutic modality or regimen. Disease subtype classifiers further find use in clinical trial design, to tailor patient recruitment to the mechanism of action of the investigational drug.

In various embodiments, the invention provides a method for generating a classifier for evaluating a subject for one or more biological conditions. The method comprises providing sRNA sequence data comprising a compilation of the distinct sRNA sequences that are present across a set of discovery samples (e.g., a training set), and selecting candidate sRNA sequences whose presence or absence, or abundance, is correlative with the presence, absence, stage, or other feature(s) of a biological condition(s) of interest. The set of discovery samples will generally comprise samples representing the presence or absence of one or more biological conditions of interest, and may further comprise non-disease controls. After reduction of candidate sRNA sequences according to embodiments of the invention (as described below), a classifier is then trained using various machine learning models, e.g., using the presence or absence, or in some embodiments the abundance, of the candidate sRNA sequences across a training set along with sample metadata comprising biological condition labels. The classifier in accordance with this aspect will comprise sRNA features for evaluating a subject's sample for the presence and/or absence of the biological conditions.

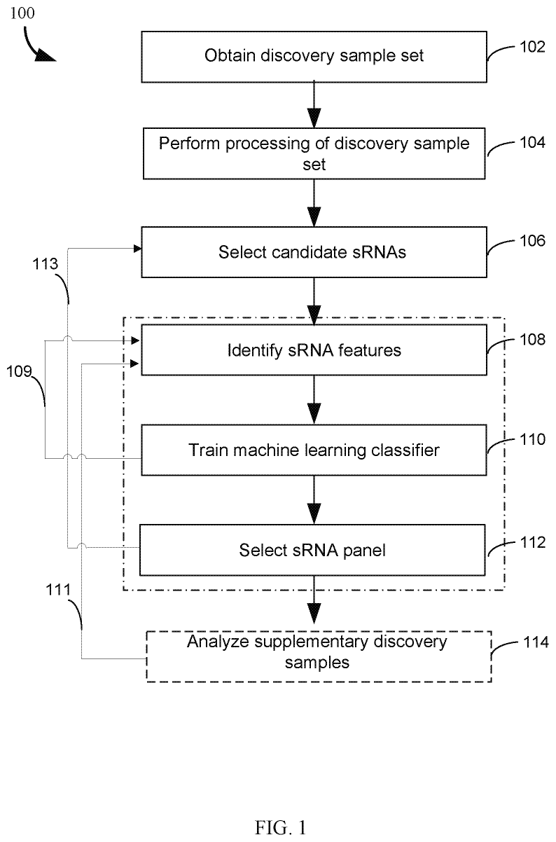

FIG. 1 illustrates schematically a method 100 of generating a classifier in accordance with some embodiments. The method 100 can be performed, at least in part, in a suitable system that in some implementations includes one or more central processing units CPU(s) (also referred to as processors), one or more graphical processing units, one or more network interfaces, a user interface, a non-persistent memory, a persistent memory, and one or more communication buses for interconnecting these components. The one or more communication buses optionally include circuitry (sometimes called a chipset) that interconnects and controls communications between system components. The non-persistent memory typically includes high-speed random access memory, such as DRAM, SRAM, DDR RAM, ROM, EEPROM, flash memory, whereas the persistent memory typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices.

The persistent memory optionally includes one or more storage devices remotely located from the CPU(s). The persistent memory, and the non-volatile memory device(s) within the non-persistent memory, comprise non-transitory computer readable storage medium. In some implementations, the non-persistent memory or alternatively the non-transitory computer readable storage medium stores (sometimes in conjunction with the persistent memory) programs, modules and data structures that are used to implement the method 100. The programs, modules and data structures can include an optional operating system (which includes procedures for handling various basic system services and for performing hardware dependent tasks); an optional network communication module (or instructions) for connecting the system with other devices, or a communication network; and other modules. For example, one or more training data sets can be stored in memory of the system. The modules, data, or programs (e.g., sets of instructions) need not be implemented as separate software programs, procedures, datasets, or modules, and thus various subsets of these modules and data may be combined or otherwise re-arranged in various implementations.

At block 102 of FIG. 1, a discovery sample set can be obtained. The discovery sample set can be obtained from any suitable source, including any one or more studies providing patient samples with matched sRNA sequence data. The set of discovery samples may comprise samples representing the presence or absence of one or more biological conditions of interest, and may further comprise non-disease controls.

As used herein, a “discovery set” or “discovery sample set” comprises a set of samples representing one or more biological conditions of interest, as well as in various embodiments, controls that do not represent any of the biological conditions of interest (non-disease controls). In some embodiments, the discovery samples are from a common tissue, and the biological conditions of interest have a common phenotype or pathology. Exemplary phenotypes or pathologies that may define the biological condition(s) of interest are not limited, but may include one or more of: cancerous malignancies; malignancy invasion; dementia; cognitive test scores; beta-amyloid protein deposits; tau tangles; movement control or tremor; neurodegeneration; demyelination; anxiety, depression, or bipolar disorder; headache or fatigue; insomnia; chronic tissue inflammation; vasculitis; vascular permeability; irritable bowel syndrome, which may include abdominal pain, diarrhea, constipation, fatigue, and/or weight loss; muscle or joint pain or fatigue; gastrointestinal permeability; muscle atrophy; autoimmunity; tissue fibrosis; disorders of physical, mental, or social development; lysosomal storage abnormality; glycogen accumulation; uncontrolled cell proliferation; cell or tissue necrosis or apoptosis; fatty liver or hepatitis; chronic kidney disease; neutrophilia or neutropenia; bone remodeling abnormalities including abnormal osteogenesis or bone resorption; insulin resistance; hypertension or hypotension; vasoconstriction; pathological angiogenesis or lymphangiogenesis; hypercholesterolemia; metabolic disease or obesity; coronary artery disease; congestive heart failure; drug response or drug toxicity; among others. In some embodiments, the discovery set is randomized into a training set and testing set for selecting candidate sRNAs and machine learning, as further described herein.

In some embodiments, the discovery set includes samples representing a biological condition of interest and obtained from patients that receive disparate therapeutic interventions or who have disparate responses to a therapeutic intervention. In such embodiments, the samples can be labeled for the particular therapeutic intervention, and/or the effectiveness or toxicity of the therapeutic intervention.

In various embodiments, samples in the set of discovery samples represent (e.g., are labeled for) the presence and absence of at least two biological conditions, or at least three biological conditions, or at least five biological conditions, and which share a common phenotype or pathology. In some embodiments, the set of discovery samples represents the presence and absence of at least four, at least five, at least seven, or at least ten biological conditions. In some embodiments, the discovery samples represent the presence and absence of from three to ten or from three to five biological conditions sharing a common phenotype or pathology.

In some embodiments, the set of discovery samples represents at least one biological condition that is suspected of having two or more distinct disease subtypes. As used herein, “disease subtype” means a collection of biological conditions that manifest with similar disease symptoms, but which may involve distinct sRNA biogenesis, disparate or distinguishable aberrant or dysregulated biological pathways, and/or which may require different modes of treatment. According to this disclosure, without intending to be bound by theory, it is believed that many complex diseases are actually a heterogeneous collection of diseases that can be meaningfully distinguished based on analysis of sRNA biogenesis. In some embodiments, the invention identifies these disease subtypes from discovery sample sets that are otherwise considered pathologically similar.

In various embodiments, the set of discovery samples comprise solid tissue samples, biological fluid samples, or cultured cells. For example, biological fluid samples may be blood, serum, plasma, cerebrospinal fluid, urine, or saliva. In some embodiments, the set of discovery samples are solid tissue biopsies (e.g., of the diseased tissue) or autopsy samples. In some embodiments, the discovery set comprises cancer cell cultures, which may be primary cultures or immortalized cell lines in some embodiments.

In various embodiments, the discovery sample set (or a training set) includes at least 50 samples, or at least 100 samples; including at least 10 samples or at least 20 samples, or at least 50 samples that are positive for each of the biological conditions of interest. In some embodiments, the discovery sample set comprises at least 25 non-disease or healthy controls, or at least 50 non-disease or healthy controls, or at least 100 non-disease or healthy controls.

The discovery set need not be sourced from a single study, and in some embodiments, it is preferred that the discovery set be sourced from separate studies to control for pre-analytical variables e.g., extraction of nucleic acid, sRNA library preparation and next generation sequencing. The term “separate studies” requires one or more of: collection of biological samples at a distinct location (e.g., separate institution); or extraction of nucleic acid or sRNA at a distinct location, and optionally using a different nucleic acid or sRNA extraction protocol or reagent from at least one other location; and sRNA sequencing library preparation and/or sequencing at a distinct location, and optionally using a different sRNA sequencing library preparation and/or sequencing protocol from at least one other location. In some embodiments, the separate studies involve sourcing or processing of tissue and/or sequencing at different geographies (e.g., at least two different countries or continents). In these embodiments, the separate procurement, processing, or sequencing provides added diversity of research protocols, and may also provide patient genetic or ethnic variation. In some embodiments, supplemental discovery samples are subsequently employed for feature reduction, as described herein.

In various embodiments, the discovery set samples are labeled as positive or negative for the one or more biological conditions of interest. In such embodiments, the invention involves identifying sRNA features for classifying samples using supervised machine learning models. In these embodiments, the invention provides classifiers for accurately classifying biological conditions that may present with similar symptoms, including at early stages of disease. Examples include CNS disorders that present with dementia or tremors, disorders that present with gastrointestinal inflammation, disorders that present with organ or tissue inflammation or fibrosis (e.g., idiopathic pulmonary fibrosis), disorders characterized by neoplasia or cell malignancy, among many others. Other disease phenotypes that may be shared across several distinct disease conditions are provided elsewhere herein.

In still other embodiments, the discovery set samples represent samples of at least one complex disease and non-disease controls. For example, the complex disease may involve one or more disease subtypes that are not labeled or are only partially labeled in the discovery set. In some embodiments, the method described herein identifies, potentially for the first time, disease subtypes. In these embodiments, the invention identifies sRNA features for classifying samples for the presence or absence of such disease subtype(s) using unsupervised or semi-supervised machine learning. Thus, even where surrogate markers are unavailable for labeling samples or where pathologist evaluations are insufficient to distinguish distinct disease subtypes, the presence or absence, or relative abundance, of sRNA sequences in panels identified by supervised machine learning in accordance with embodiments of the invention, provide surprisingly effective means to subtype samples of a complex disease. In some embodiments, the invention as described herein is used to identify and classify these disease subtypes from discovery sample sets that are otherwise considered pathologically similar.

Referring back to FIG. 1, in some embodiments, as shown at block 104, the sRNA sequencing data in the discovery sample set is processed, which involves adapter trimming. In some embodiments, the adapter trimming can be performed, for example, as described in PCT/US2018/014856, the entire contents of which are hereby incorporated by reference.

In some embodiments of the present disclosure, sRNA sequence data for the discovery sample set is provided. The sRNA sequence data is processed by trimming 5′ and 3′ sequencing adapters from sRNA sequence reads to identify the 5′ and 3′ variations present. These distinct variations are not consolidated based on a reference sequence or genetic locus, which is the conventional approach for analyzing miRNA. Accordingly, the sRNA sequence data from the discovery set involves the compilation of the distinct sRNA sequences (i.e., isoforms) in each sample across the discovery samples.

In order to identify variations at the 5′ and 3′ ends of the sRNAs, a user-defined sequencing adapter may be trimmed from the raw sRNA sequence reads, using, e.g., a suitable computational module (e.g., a software program). The adapter is defined by the user, based on the sequencing platform. By removing the adapter sequence, sRNA isoforms can be identified and quantified in samples. For example, in some embodiments a software program searches for regular expressions corresponding to a user-defined 3′ adapter and deletes them from the raw sRNA sequence reads.

In some embodiments, the regular expression of the user-defined 3′ adapter includes some number of “wild cards.” A wild-card is defined as being any one of the 4 deoxyribonucleic acids: (A) adenine, (T) thymine, (G) guanine, or (C) cytosine. However, the first nucleotide at the 5′ end of the user-specified 3′ adaptor sequence is not altered (e.g., not considered an insertion or deletion or otherwise subject to wild-card change), thus preserving sRNA sequences at the junction where the 3′ terminal nucleotide of the sRNA is ligated to the 5′ terminal nucleotide of the 3′ adapter. If the 5′ terminal nucleotide of the user-specified 3′ adapter does not correspond with what the user has specified, the 3′ adapter sequence is not trimmed, but can be independently verified, if needed. In some embodiments, sRNAs having a length of at least 17 nucleotides (after trimming) are considered for analysis. In some embodiments, sRNAs having a length of no more than about 75 nucleotides, or no more than about 50 nucleotides, or no more than about 43 nucleotides are considered for analysis.

In some embodiments, the presence or absence, or abundance, of the distinct sRNA sequences is determined. In such embodiments, sRNA sequences can be normalized to one or more endogenous sRNA controls or an exogenous (i.e., “spike-in”) sRNA control. In some embodiments, a spike-in can be (1) a synthetic oligonucleotide, (2) an equimolar pool of synthetic oligonucleotides, or (3) a pool of synthetic oligonucleotides mixed at increasing concentrations. In each embodiment, a spike-in is added to a sample before 5′ and 3′ adapter ligation. In each of the above cases, the oligonucleotides are synthesized with a 5′ phosphate and 3′ hydroxyl to mimic endogenous sRNAs.

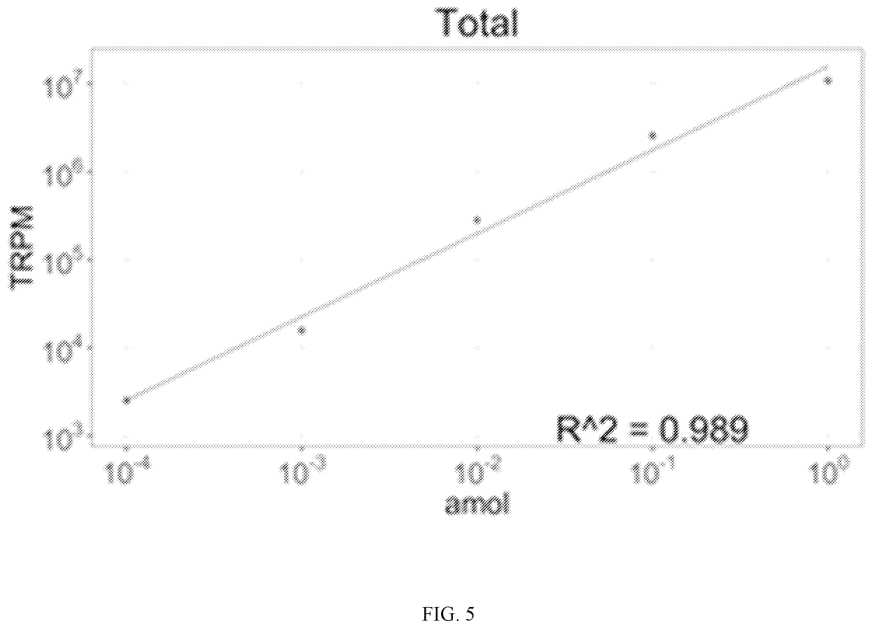

In some embodiments, as described in more detail in Example 2 (FIG. 5), a pool of a certain number of exogenous, oligonucleotides synthesized with a 5′ phosphate and 3′ hydroxyl are combined at various concentrations, and can be added to each sample prior to 5′ and 3′ adaptor ligation.

sRNA sequencing enriches and sequences small RNA species, such as microRNA (miRNA), Piwi-interacting RNA (piRNA), small interfering RNA (siRNA), vault RNA (vtRNA), small nucleolar RNA (snoRNA), transfer RNA-derived small RNAs (tsRNA), ribosomal RNA-derived small RNA fragments (rsRNA), small rRNA-derived RNA (srRNA), and small nuclear RNA (U-RNA). For example, in providing the sRNA sequencing data, input material may be enriched for small RNAs. Sequence library construction is performed with sRNA-enriched material using any of several processes or commercially-available kits depending on the high-throughput sequencing platform being employed. Generally, sRNA sequencing library preparation comprises isolating total RNA from samples, size fractionation, ligation of sequencing adapters, reverse transcription and PCR amplification, and DNA sequencing.

More particularly, in some embodiments, in a given sample all the RNA (i.e., total RNA) is extracted and isolated. The small RNAs are isolated by size fractionation, for example, by running the isolated RNA on a denaturing polyacrylamide gel or using any of a variety of commercially available kits. A ligation step then adds adapters to both ends of the small RNAs, which act as primer binding sites during reverse transcription and PCR amplification. For example, a pre-adenylated single strand DNA 3′-adapter followed by a 5′-adapter are ligated to the small RNAs using a ligating enzyme, such as T4 RNA Ligase 2 Truncated (T4 Rn12tr K227Q). The adaptors are designed to capture small RNAs with a 5′-phosphate and 3′-hydroxyl group, characteristic of biologically processed small RNAs (e.g., microRNAs), rather than RNA degradation products having different 5′ and 3′ end chemistry. The sRNA library is then reverse transcribed and amplified by PCR. This step converts the adaptor ligated RNAs into cDNA clones that are the template for the sequencing reaction. Primers designed with unique nucleotide index sequences can also be used in this step to create ID tags (i.e., bar codes) to facilitate library pooling and multiplex sequencing.

Any DNA sequencing platform can be employed, including any next-generation sequencing platform such as pyrosequencing (e.g., 454 Life Sciences), polymerase-based sequence-by-synthesis (e.g., Illumina), or sequencing-by-ligation (e.g., ABI Solid Sequencing platform), among others.

Referring back to FIG. 1, at block 106, candidate sRNAs can be selected from the sRNAs processed at block 104. In some embodiments, candidate sRNAs are limited to one or more of miRNA isoforms, transfer RNA-derived fragment, and ribosomal RNA-derived fragments. In some embodiments, these miRNA, tRNA, and rRNA species are filtered from the sRNA sequences, and used for candidate selection. In some embodiments, one or more candidate sRNAs are isomiRs. “isomiR” refers to those sequences that have variations with respect to the reference miRNA sequence (e.g., as used by miRBase). In miRBase, each miRNA is associated with a miRNA precursor and with one or two mature miRNA (−5p and −3p). Deep sequencing detects a large amount of variability in miRNA biogenesis, meaning that from the same miRNA precursor many different sequences can be detected. There are six main variations of sRNAs: (1) 5′ alteration, where the 5′ terminal nucleotide is upstream or downstream from the referenced sRNA sequence; (2) 3′ alteration, where the 3′ terminal nucleotide is upstream or downstream from the reference sRNA sequence; (3) 5′ nucleotide addition, where nucleotides are enzymatically added to the 5′ end of the reference sRNA; (4) 3′ nucleotide addition, where nucleotides are enzymatically added to the 3′ end of the reference sRNA; and (5) nucleotide substitution, where nucleotides are altered due to a DNA variant (e.g., single nucleotide polymorphisms, insertions or deletions); (6) nucleotide editing, where nucleotides are altered due to enzymatic altering of one or more nucleotide bases in a miRNA precursor or mature miRNA or other sRNA. In some embodiments, inclusion of isomiRs are limited to 5′ and 3′ variants, but not substitutions or “swaps”. In some embodiments, intergenic mapping miRNAs are disallowed in the candidate sRNA selection process.

In some embodiments, one or more candidate sRNA variants are transfer RNA-derived fragments, without swaps. In some embodiments, one or more candidate sRNA variants are ribosomal RNA-derived fragments, without swaps.

In accordance with various embodiments, at block 106 of FIG. 1, sRNA sequence data from the discovery set is used to select candidate sRNA sequences for machine learning. In order to improve machine learning, the distinct sRNA sequences, which may be around the order of 100 million distinct sequences in the discovery set, are filtered to several thousand candidate sRNAs using pre-selection criteria. For example, in some embodiments, no more than about 100,000 sRNA sequences are selected for machine learning analysis, or no more than about 50,000 sRNA sequences, or no more than about 10,000 sRNA sequences, or no more than about 5,000 sRNA sequences, or no more than about 2,000 sRNA sequences are selected for training the disease classifier using machine learning models. In various embodiments, at least about 1000, or at least about 2000, or at least about 5000, or at least about 10,000 candidate sRNAs are preselected for supervised machine learning. In some embodiments, from about 2,500 to about 60,000 sRNAs sequences are preselected for training the disease classifier.

At block 106 of FIG. 1, in some embodiments, after the sRNA sequence data from the discovery set is processed, candidate sRNA sequences are selected from the sRNA sequence data. The candidate sRNA sequences can be selected based on the degree to which their presence, absence, or abundance correlates to the presence or absence of a biological condition of interest, e.g., as compared to other condition(s) or non-disease controls present in the discovery set. In some embodiments, at least one candidate sRNA sequence is only present in discovery samples (e.g., of the training set) that are positive for a biological condition of interest, and absent in all other discovery samples. In some embodiments, at least five, or at least ten, or at least twenty candidate sRNA sequences are selected that are present only in samples that are positive or negative for the biological condition of interest, and absent in all other discovery samples. In some embodiments, sRNAs are filtered for those that are present in disease samples at a defined frequency threshold (and absent in all other samples of at least one other class (e.g., healthy control or other biological condition)). For example, sRNAs may be filtered for those that are present in at least about 5%, or at least about 10%, or at least about 15%, or at least about 20%, or at least about 25% of the samples that are positive for a biological condition of interest. In addition, sRNA sequences can be filtered for those that are present in control samples at a defined frequency threshold (and absent in all samples of at least one biological condition class). For example, sRNAs may be filtered for those that are present in at least about 5%, or at least about 10%, or at least about 15%, or at least about 20%, or at least about 25% of the samples that are healthy (non-disease) controls. sRNA markers that are identified as present in samples of one class, but absent in all samples of at least one other class, are sometimes referred to herein as “binary” markers.

In various embodiments, candidate sRNA sequences are selected that individually predict, by their presence or absence, for a biological condition of interest in the discovery set, and particularly the set of samples in the training group. For example, candidate sRNA sequences can be selected whose presence or absence is predictive of a biological condition of interest, and with a p-value of at least 0.01 in the training group. In some embodiments, at least one candidate sRNA sequence (e.g., at least 2, 3, 4, or 5 candidate sRNA sequences) is selected whose presence or absence is predictive of a biological condition of interest, and with a p-value of at least 0.0001 in the training group. In some embodiments, at least one candidate sRNA sequence (e.g., at least 2, 3, 4, or 5 candidate sRNA sequences) is selected whose presence or absence is predictive of a biological condition of interest, and with a p-value of at least 0.000001 in the training group. In some embodiments, at least one candidate sRNA sequence (e.g., at least 2, 3, 4, or 5 candidate sRNA sequences) is selected whose presence or absence is predictive of a biological condition of interest, and with a p-value of at least 0.00000001 in the training group. In some embodiments, at least one candidate sRNA sequence (e.g., at least 2, 3, 4, or 5 candidate sRNA sequences) is selected whose presence or absence is predictive of a biological condition of interest, and with a p-value of at least 0.0000000001 in the training group. In various embodiments, such candidate sRNA sequences are selected for each biological condition of interest. That is, candidate sRNAs include sequences selected individually for their predictive power in determining the presence or absence of at least one biological condition, against other biological conditions represented in the discovery set and/or non-disease controls.

In some embodiments, preselection is implemented at least in-part by selecting a frequency threshold for candidate sRNAs in the training group. That is, candidate sRNAs must be present at a minimum frequency in a particular class, but below a designated frequency threshold in at least one other class (in the training group). For example, the candidate sRNAs may be present in at least about 50% of samples in a particular class, or at least about 40% of samples in a particular class, or at least about 25% of samples in a particular class, or at least about 20% of samples in a particular class, or at least about 15% of samples in a particular class, or at least about 10% of samples in a particular class (in the training group), or at least about 5% of samples in a particular class. In some embodiments, the candidate sRNA will meet this threshold requirement for each independent study represented in the class. With respect to such candidate sRNAs, these will be present below a threshold in at least one other class, such as less than about 15% of samples in at least one other class, or less than about 10% of samples in at least one other class, or less than about 5% of samples in at least one other class in the training group. In some embodiments, the candidate sRNAs are absent in all samples of at least one other class in the training group.

In some embodiments, candidate sRNA sequences are selected from the sequence data, based on the degree to which their abundance correlates to the presence or absence of a biological condition of interest, e.g., as compared to other condition(s) or non-disease controls present in the discovery set. In some embodiments, at least one candidate sRNA sequence has an abundance level that is indicative of the presence or absence of a biological condition of interest (e.g., the abundance is above or below a certain threshold). In some embodiments, the difference in relative abundance between disease and non-disease samples is at least about 5 fold, or at least about 10 fold, or at least about 100 fold, or at least about 1000 fold, or at least about 10,000 fold. sRNA markers that are selected based on differential abundance levels between at least two classes, are sometimes referred to herein as “differentially expressed” markers.

In some embodiments, candidate sRNA sequences are selected that individually predict, based on their abundance, the presence or absence of a biological condition of interest. For example, candidate sRNA sequences can be selected whose abundance is predictive of the presence or absence of a biological condition of interest, and with a p-value of at least 0.01 in the training group. In some embodiments, at least one candidate sRNA sequence (e.g., at least 2, 3, 4, or 5 candidate sRNA sequences) is selected whose abundance is predictive of the presence or absence of a biological condition of interest, and with a p-value of at least 0.0001 in the training group. In some embodiments, at least one candidate sRNA sequence (e.g., at least 2, 3, 4, or 5 candidate sRNA sequences) is selected whose abundance is predictive of the presence or absence of a biological condition of interest, and with a p-value of at least 0.000001 in the training group. In some embodiments, at least one candidate sRNA sequence (e.g., at least 2, 3, 4, or 5 candidate sRNA sequences) is selected whose abundance is predictive of the presence or absence of a biological condition of interest, and with a p-value of at least 0.00000001 in the training group. In some embodiments, at least one candidate sRNA sequence (e.g., at least 2, 3, 4, or 5 candidate sRNA sequences) is selected whose abundance is predictive of the presence or absence of a biological condition of interest, and with a p-value of at least 0.0000000001 in the training group. In various embodiments, such candidate sRNA sequences are selected for each biological condition of interest. That is, candidate sRNAs include sequences selected individually for their predictive power in determining the presence of at least one biological condition, against other biological conditions represented in the discovery set and/or non-disease controls in the training group.

In some embodiments, preselection for sRNAs that are increased in abundance is implemented at least in-part by selecting a frequency threshold for candidate sRNAs. That is, candidate sRNAs must be significantly higher or lower in abundance at a minimum frequency in a particular class, as compared to the relative level of abundance (e.g., mean or median) observed in samples of at least one other class in the training group. For example, the candidate sRNAs may be significantly higher or lower in relative abundance in at least about 50% of samples in a particular class, or at least about 40% of samples in a particular class, or at least about 25% of samples in a particular class, or at least about 20% of samples in a particular class, or at least about 15% of samples in a particular class, or at least about 10% of samples in a particular class, or at least 5% of samples in a particular class (as compared to the relative abundance of the sRNA observed in at least one other class) in the training group. In some embodiments, the candidate sRNA will meet this threshold requirement for each independent study represented in the class in the training group. With respect to such candidate sRNAs, the change in relative abundance is observed below a frequency threshold in the at least one other class, such as less than about 15% of samples in at least one other class, or less than about 10% of samples in at least one other class, or less than about 5% of samples in at least one other class in the training group. In some embodiments, the candidate sRNAs have a statistically significant change in relative abundance in samples of the particular class that is not observed in any sample of at least one other class in the training group.

The number of candidate sRNAs can be further reduced using, for example, linear or logistic regression models.

In some embodiments, the set of discovery samples are further labeled for stage, grade, or other characteristic(s) of the biological condition of interest. In these embodiments, candidate sRNAs may be selected whose read counts correlate (e.g., directly) with disease activity, such as, for example, disease stage or grade. For example, as disease stage or grade progresses, candidate sRNA sequences can be selected that show higher read counts. That is, average read counts increase in later stages of the disease, or with higher disease activity. Alternatively, as the disease severity decreases (e.g., in a treatment group), candidate sRNA sequences can be selected that show lower read counts in treated subjects. Thus, in some embodiments, at least one, two, three, four, or five candidate sRNA sequences are selected whose presence or abundance is predictive of a biological condition represented by samples in the discovery set, and whose read count is correlative with disease stage or grade of the disease in such samples. Where average read counts are desirable to select candidate sRNA sequences, the sRNA sequences can be determined using one or more of endogenous sRNA and/or spike-in normalization controls, as described, e.g., in Example 2 below.

In various embodiments, sRNA families are identified that have increased sequence diversity in a biological condition of interest. sRNA sequences within these sRNA families are selected as candidate sRNA sequences. For example, in some embodiments, sRNA families can be identified in which sequence variation increases in a disease condition and/or increases with severity of a disease condition, and/or which variation may normalize or be ameliorated in response to a therapeutic regimen. For example, sRNA pre-selection can involve grouping sRNA isoforms (such as isomiRs) into “families” based on biologically relevant sequence features. In some embodiments, the sequence feature is a miRNA “seed sequence,” which generally includes nucleotides 2 to 8 from the 5′ end of the annotated sRNA. In some embodiments, the sequence feature is a single nucleotide polymorphism or INDEL. These sRNA families are evaluated for variation at the 5′ and 3′ ends. For example, variations can include 5′ and/or 3′ variation including templated and/or non-templated nucleotide additions, or 5′ and/or 3′ trimming, and which can be correlative with the presence of disease or disease activity. These entire families or predictive variants within the family can be selected as candidates for machine learning. In some embodiments, these families include at least one sRNA sequence that is unique in the biological condition of interest.

In some embodiments, linear or logistic regression models are weighted for sRNA isoforms (isomiRs) with a common seed sequence or sRNAs with properties associated with the presence in exosomes (such as 3′ non-templated addition of nucleotide(s), e.g., U(s)). In some embodiments, miRNAs with common seed regions are aggregated (e.g., using a preselection filter) during candidate sRNA reduction.

Other parameters can be used to aid selection of candidate sRNA sequences. For example, the set of discovery samples can be sourced from at least two separate studies as described elsewhere herein, which in some embodiments includes sourcing from at least two different institutions, countries, or continents. In these embodiments, the selected candidate sRNA sequences are each present in at least one sample from each study (or above a frequency threshold in each study), thereby reducing the likelihood that the sequence is a study artifact. The separate studies may involve collection of biological samples at distinct locations, or extraction of nucleic acid or sRNA at distinct locations, or sequencing library preparation and/or sequencing at distinct locations. In some embodiments, the distinct studies employ differing nucleic acid or sRNA extraction protocols or distinct sequencing library preparation protocols and/or sequencing protocols.

In various embodiments, sRNA sequences are preselected based on a threshold average read count in the discovery set. For example, the selected sRNA sequences may have an average read count of at least 0.1 trimmed reads per million reads. In some embodiments, sRNA sequences are selected with a read count above a designated floor, and below designated ceiling. In some embodiments, a sequencing depth is a sliding scale based on the biological matrix. For example, solid tissue samples may be sequenced at 5,000,000 to 15,000,000 million reads per sample; cerebrospinal fluid, serum and plasma samples may be sequenced at 15,000,000 to 35,000,000 million reads per sample; and PAXgene (whole blood) samples may be sequenced at 35,000,000 to 55,000,000 million reads per sample. By sequencing at a higher depth, the method takes into account dilution of sRNAs as they exit tissue and make their way into the periphery.

In various embodiments, candidate sRNA sequences are selected based on their ability to map to the human genome.

Referring back to FIG. 1, once the candidate sRNAs are selected from a training set (including reduction to the desired number of candidate sRNAs for machine learning), sRNA features can be identified (block 108) for training a classifier. Various feature selection or extraction approaches can be used to select features appropriate for a machine learning classifier. In some embodiments, the features can be in the form of processed data—e.g., polynucleotide sequences of sRNAs selected at block 106 (which are previously processed, e.g., by adapter trimming). Furthermore, in some embodiments, features can be generated that are multidimensional data points. In order to reduce computational burden, dimension of such features can be reduced, for example, using a statistical feature selection or feature extraction procedure known in the art, e.g., principal component analysis, non-negative matrix factorization, ROC curve for feature ranking, kernel PCA, graph-based kernel PCA, UMAP, linear discriminant analysis, generalized discriminant analysis. Similarly, in some embodiments, a machine learning technique is used to reduce the number of dimensions of the multidimensional data points, e.g., a neural network, a convolutional neural network, an autoencoder, a support vector machine, a Bayesian network, or a genetic algorithm.

In some embodiments, with reference to block 110, after the sRNA features are selected, a machine learning classifier can be trained, using one or more machine learning approaches. In some embodiments, the classifier is configured to classify samples based on the presence or absence, or abundance, of a panel of sRNA sequences (from the candidate sRNAs). In some embodiments, a desired panel size can be selected. Generally, the size of the panel can be larger where there are more disease classes. For example, in some embodiments, the panel contains from about 1 to about 50,000 sRNA sequences, such as about 1 to about 200 sRNA sequences per class, or from about 4 to about 100 sRNA sequences per class, or from about 4 to about 50 sRNA sequences per class. In some embodiments, the panel comprises from about 10 to about 100 sRNA sequence per class, or from about 10 to about 50 sRNA sequences per class, or from about 10 to about 40 or from about 10 to about 30 sRNA sequences per class. In some embodiments, the panel contains from about 50 to about 150 sRNA sequences, or from about 50 to about 100 sRNA sequences per class. In some embodiments, a minimal or reduced panel is selected having a total panel of from 1 to about 500 sRNA sequences, or from 1 to about 200 sRNA sequences, or from about 4 to about 100 sRNA sequences, or from about 4 to about 50 sRNA sequences, or from about 10 to about 100 sRNA sequence, or from about 10 to about 50 sRNA sequences, or from about 10 to about 40 sRNA sequences, or from about 10 to about 30 sRNA sequences, or from about 50 to about 150 sRNA sequences, or from about 50 to about 100 sRNA sequences. In some embodiments, the panel contains no more than about 100 sRNA sequences, or no more than 96 sRNA sequences, or no more than 75 sRNA sequences, or no more than 50 sRNA sequence.

In some embodiments, the classifier is based on a support vector machine algorithm, a decision tree algorithm, an unsupervised clustering algorithm, a supervised clustering algorithm, a logistic regression algorithm, a mixture model, a hidden Markov model, or a neural network algorithm.

In various embodiments, the classifier is trained using one or more of supervised, unsupervised, semi-supervised machine learning models such as, for example: parametric/non-parametric distance measures, logistic regression, support vector machines, decision trees, random forests, neural networks, probit regression, Fisher's linear discriminant, Naive Bayes classifier, perceptron, quadratic classifiers, kernel estimation, k-nearest neighbor, learning vector quantization, and PCA. For example, in some embodiments, the classifier is trained using at least linear support vector machine.

In some embodiments, the classifier is an unsupervised clustering model. In some embodiments, the classifier is a supervised clustering model. Clustering is described at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem involves finding natural groupings in a dataset. To identify natural groupings, two issues are addressed. First, a way to measure similarity (or dissimilarity) between two samples is determined. This metric (e.g., similarity measure) is used to ensure that the samples in one cluster are more like one another than they are to samples in other clusters. Second, a mechanism for partitioning the data into clusters using the similarity measure is determined. To begin a clustering investigation one can define a distance function and compute the matrix of distances between all pairs of samples in the training set. If distance is a good measure of similarity, then the distance between reference entities in the same cluster will be significantly less than the distance between the reference entities in different clusters. However, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.”

Once a method for measuring “similarity” or “dissimilarity” between points in a dataset has been selected, clustering requires a criterion function that measures the clustering quality of any partition of the data. Partitions of the data set that extremize the criterion function are used to cluster the data. More information on clustering techniques can be found in Kaufman and Rousseeuw, 1990, Finding Groups in Data: An Introduction to Cluster Analysis, Wiley, N.Y., N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J., each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering where no preconceived notion of what clusters should form when the training set is clustered are imposed. In some embodiments, the unsupervised clustering can be used to identify disease subtypes, such that meaningful patterns can be discovered within the sRNA data and utilized in research and clinical applications.

In some embodiments, the classifier is a regression model, such as the multi-category logit models described in Agresti, An Introduction to Categorical Data Analysis, 1996, John Wiley & Sons, Inc., New York, Chapter 8, which is hereby incorporated by reference in its entirety. In some embodiments, the classifier makes use of a regression model disclosed in Hastie et al., 2001, The Elements of Statistical Learning, Springer-Verlag, New York.

In some embodiments, the classifier is a Naive Bayes algorithm, such as the tool developed by Rosen et al. to deal with metagenomic reads (See, Bioinformatics 27(1):127-129, 2011). In some embodiments, the classifier is a nearest neighbor algorithm, such as the non-parametric methods described by Kamvar et al., Front Genetics 6:208 doi: 10.3389/fgene.2015.00208, 2015). In some embodiments, the classifier is a mixture model, such as that described in McLachlan et al., Bioinformatics 18(3):413-422, 2002. In some embodiments, in particular those embodiments including a temporal component, the classifier is a hidden Markov model such as described by Schliep et al., 2003, Bioinformatics 19(1):i255-i263.

Principal component analysis (PCA) algorithms are described in Jolliffe, 1986, Principal Component Analysis, Springer, N.Y., which is hereby incorporated by reference. PCA is also described in Draghici, 2003, Data Analysis Tools for DNA Microarrays, Chapman & Hall/CRC, which is hereby incorporated by reference. Principal components (PCs) are uncorrelated and are ordered such that the k^{th} PC has the k^{th} largest variance among PCs. The k^{th} PC can be interpreted as the direction that maximizes the variation of the projections of the data points such that it is orthogonal to the first k-1 PCs. The first few PCs capture most of the variation in a training set. In contrast, the last few PCs are often assumed to capture only the residual ‘noise’ in the training set.

SVM algorithms are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, N.Y; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, N.Y.; and Furey et al., 2000, Bioinformatics 16, 906-914, each of which is hereby incorporated by reference in its entirety. When used for classification, SVMs separate a given set of binary labeled data training set with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

In some embodiments, feature selection and training the machine learning classifier (at blocks 108 and 110 of FIG. 1, respectively) can be part of the same processing, such that the classifier is used for cross-validation and selection of appropriate features, as schematically shown by an arrow 109 in FIG. 1. The trained machine learning classifier can be used to select an sRNA panel, as shown at block 112 of FIG. 1. It should be appreciated that the training of the machine learning classifier and selection of the sRNA panel can be part of the same process. Also, the list of sRNAs included in the sRNA panel can be adjusted iteratively, as shown schematically by an arrow 113 in FIG. 1.

In some embodiments, referring again to block 110 of FIG. 1, to train the machine learning classifier, 10% to 90% of the samples are randomized into a training set. Pre-selection is used to select, for example, 2,400 to 60,000 small RNA features from the training set with a minimum TRPM (trimmed reads per million) of 0.1 to 100. The sRNA feature set can be reduced to 1 to 1,000 sRNA features per class using regression models. The final set of sRNA features is used to test on the remaining 90% to 10% of the samples using linear regression or a support vector machine with a threshold of 51% to 100% confidence interval to classify a sample. Accuracy is calculated using standard Receiving Operator Characteristics to calculate true positive, false positive, true negative and false negative rates, overall accuracy and area under the curve. The term “ROC” or “ROC curve,” refers to a Receiver Operator Characteristic curve. A ROC curve can be a graphical representation of the performance of a binary classifier system. For any given method, a ROC curve can be generated by plotting the sensitivity against the specificity at various threshold settings. Furthermore, provided at least one of three parameters (e.g., sensitivity, specificity, and the threshold setting), a ROC curve can determine the value or expected value for any unknown parameter. The unknown parameter can be determined using a curve fitted to a ROC curve. For example, provided the presence/absence or abundance of a panel of sRNAs in a sample, the expected sensitivity and/or specificity of a test can be determined. The term “AUC” or “ROC-AUC” can refer to the area under a receiver operator characteristic curve. This metric can provide a measure of diagnostic utility of a method, taking into account both the sensitivity and specificity of the method. A ROC-AUC can range from 0.5 to 1.0, where a value closer to 0.5 can indicate a method has limited diagnostic utility (e.g., lower sensitivity and/or specificity) and a value closer to 1.0 indicates the method has greater diagnostic utility (e.g., higher sensitivity and/or specificity). See, e.g., Pepe et al., 2004, “Limitations of the Odds Ratio in Gauging the Performance of a Diagnostic, Prognostic, or Screening Marker,” Am. J. Epidemiol 159 (9): 882-890, which is incorporated herein by reference in its entirety. Additional approaches for characterizing diagnostic utility include using likelihood functions, odds ratios, information theory, predictive values, calibration (including goodness-of-fit), and reclassification measurements. Examples of the approaches are summarized, e.g., in Cook, “Use and Misuse of the Receiver Operating Characteristic Curve in Risk Prediction,” Circulation 2007, 115: 928-935, which is incorporated herein by reference in its entirety. In embodiments of the present disclosure, classifiers can be binary classifiers (i.e., classify among two classes representing, e.g., conditions), or may classify among three, four, five, or more biological conditions. In some embodiments, the classifier can classify among at least three, at least five, at least ten, at least fifteen, at least twenty, at least twenty-five, at least thirty, or at least thirty-five biological conditions.

5 In some embodiments, as shown at block 114 in FIG. 1, after training of the machine learning classifier, supplemental discovery samples can be evaluated to reduce a number of classifier features or number of sRNAs in the panel (see arrow 111 in FIG. 1). For example, the value of the classifier features with respect to classifying supplemental samples can be used to weight individual features or reduce the feature set. In some embodiments, at least 100 sRNA sequences are included in the original feature set based on the discovery samples, and this feature set is reduced to less than 75 or less than 50, or less than 20 using sRNA sequence data from supplemental samples. In various embodiments, using the supplemental discovery samples, the sRNA panel is generally reduced by at least 10%, or at least 25%, or at least 50% in some embodiments. In various embodiments, supplemental discovery samples include samples with distinct collection criteria, with respect to the discovery set, such as collection of biological samples at distinct locations, or separate extraction of nucleic acid or sRNA at distinct locations, or separate sRNA sequencing library preparation and/or sequencing at distinct locations. In some embodiments, the supplemental samples employ differing nucleic acid or sRNA extraction protocols or distinct sequencing library preparation protocols and/or sequencing protocols. It should be noted that the processing at block 114 (FIG. 1) can be performed before the sRNA panel is selected.

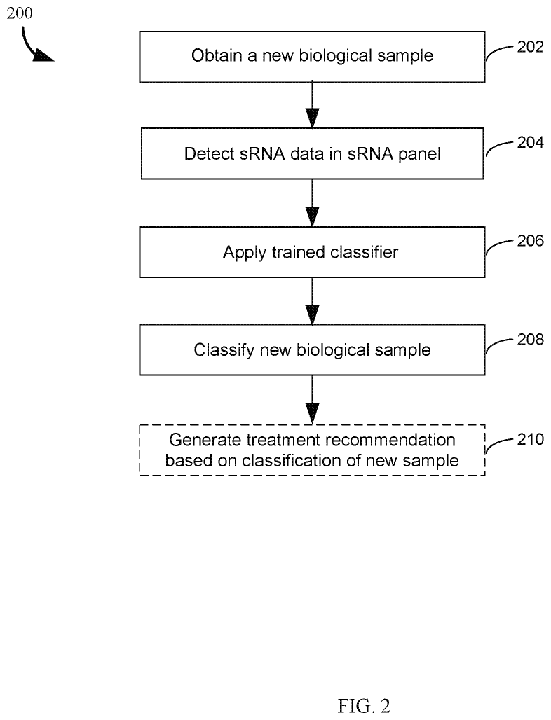

The trained machine learning classifier can be used for evaluation of independent subjects for the disease conditions or to further identify and evaluate for disease subtypes (e.g., of complex disease), by detecting the presence or absence or abundance, in a biological sample from the subject, of sRNA markers in the panel, and applying the classifier. FIG. 2 illustrates an embodiment of a method 200 of evaluating (testing) a subject for diseases or conditions, or disease subtypes, in accordance with some embodiments. At block 202, a biological sample can be obtained from a subject (e.g., a human). The biological sample can be a sample that was not used to train the machine learning classifier, and it can be referred to in some embodiments as a test sample. At block 204, sRNA data can be detected and quantified for an sRNA panel, which may involve determining the presence, absence, or abundance of the sRNAs from the biological sample in one or more sRNA panels. sRNAs may be detected and/or quantified in the sample using a molecular detection assay (such as quantitative or semi-quantitative PCR, or other approach described herein), or may be conducted by sRNA sequencing and trimming of adapter sequences from reads. sRNA sequencing can involve capture RNA sequencing (e.g., capture-enriched sRNA sequencing). Depending on a type of an sRNA panel, in some embodiments, abundance of sRNAs from the sample is determined. At block 206, the trained classifier can be applied to the detected sRNA data to assign the biological sample to a class, with reference to block 208 of FIG. 2. In some embodiments, the assignment of the biological sample to a class can be associated with a score or another measure that indicates the confidence with which the classifier assigned the biological sample to the class (i.e., predicted that the biological sample belongs to the class). Thus, in some implementations, the biological sample can be assigned to more than one class, with a corresponding probability, or another measure, computed with respect to each class. In some cases, only the assignments with associated probability values above a certain threshold can be provided by the classifier (e.g., shown on a user interface, communicate over a network, and/or otherwise outputted to a user). The threshold can be selected in various ways, including based on a user input.

Furthermore, in some embodiments, as shown in FIG. 2 (block 210), a treatment recommendation or regimen can be generated based on the results of the classification of the subject's biological sample.

Classification with respect to various biological conditions can be performed in accordance with the subject matter of the present disclosure. In some embodiments, the biological conditions for classification are conditions of the central nervous system. For example, in some embodiments, biological conditions are neurodegenerative diseases involving symptoms of dementia. In some embodiments, biological conditions are selected from Alzheimer's disease, Parkinson's disease, Huntington's disease, Mild Cognitive Impairment, Progressive Supranuclear Palsy, Frontotemporal Dementia, Lewy Body Dementia, and Vascular Dementia. In these or other embodiments, at least two biological conditions for classification are neurodegenerative diseases involving symptoms of loss of movement control. For example, in some embodiments at least two biological conditions are selected from Alzheimer's disease, Progressive Supranuclear Palsy, Hippocampal Sclerosis, Lewy Body Dementia, Parkinson's disease, Huntington's disease, Multiple Sclerosis, Amyotrophic Lateral Sclerosis, and Spinal Muscular Atrophy. In some embodiments, the biological condition(s) for classification are demyelinating diseases, which may include multiple sclerosis, optic neuritis, transverse myelitis, and neuromyelitis optica.

In some embodiments, the discovery set is labelled for disease stage, disease severity, drug responsiveness, or course of disease progression. These embodiments find particular use for evaluating biological conditions such as Alzheimer's disease, Parkinson's disease, Huntington's disease, Multiple Sclerosis, Amyotrophic Lateral Sclerosis, and Spinal Muscular Atrophy.

In still other embodiments, the biological conditions for classification are cancers of different tissue or cell origin. In these or other embodiments, the discovery set may also be labelled for drug sensitivity or drug resistance, allowing for these properties to be evaluated in a subject's sample. In some embodiments, the biological sample from the subject is a tumor or cancer cell biopsy. In still other embodiments, the biological sample is a blood, serum or plasma sample.

In some embodiments, the biological conditions for classification are inflammatory or immunological diseases. Exemplary inflammatory or immunological diseases include one or more of Systemic Lupus Erythematosus (SLE), scleroderma, autoimmune vasculitis, diabetes mellitus (type 1 or type 2), Grave's disease, Addison's disease, Sjogren's syndrome, thyroiditis, rheumatoid arthritis, myasthenia gravis, multiple sclerosis, fibromyalgia, psoriasis, Crohn's disease, ulcerative colitis, diverticular disease and celiac disease. In some embodiments, the discovery set comprises biological fluid samples such as tissue, blood, serum, plasma, or cerebrospinal fluid.

In some embodiments, the biological conditions for classification are cardiovascular diseases. In some embodiments, the discovery set is labeled for risk of an acute cardiovascular event. In such embodiments, the disease classifier provides a convenient tool for stratification of patients for risk of an acute event. In some embodiments, the cardiovascular diseases include one or more of coronary artery disease (CAD), myocardial infarction, stroke, congestive heart failure, hypertensive heart disease, cardiomyopathy, heart arrhythmia, congenital heart disease, valvular heart disease, carditis, aortic aneurysms, peripheral artery disease, and venous thrombosis.

In various embodiments, as mentioned above, the classifier identifies disease subtypes, for example, of a complex disease. In such embodiments, the entire set of discovery samples relating to a biological condition of interest (e.g., excluding non-disease controls), or a substantial number of the samples relating to a biological condition of interest (e.g., more than about 25%, or more than about 50%, or more than about 75%) are not labeled for disease subtype. In such embodiments, sRNA panels created using supervised machine learning to classify the complex disease, can be employed in unsupervised or semi-supervised machine learning approaches to identify disease subtype. In these embodiments, sRNA panels provide powerful means for cluster analysis to identify distinct disease subtypes involving distinct sRNA biogenesis patterns.

The panel of sRNAs (e.g., miRNAs) used in the subtype classifier can be used to identify different druggable targets or pathways for the distinct disease subtypes. Biological databases that find use in mapping the sRNAs to mRNA targets and pathways are described in Zou D, et al., Biological Databases for Human Research, Genomics Proteomics Bioinformatics, 13 (2015) 55-63, which is hereby incorporated by reference in its entirety. Examples include Database of Essential Genes (DEG), Kyoto Encyclopedia of Genes and Genomes (KEGG), KEGG Pathways, GeneCards, PolymiRTS (polymorphism in miRNAs and their target sites), ChIPBase, miRTarBase, miRWalk, piRNABank, Database of Interacting Protein (DIP), and Molecular Interaction Database (MINT), among others.

For example, biological pathways can be identified that involve genes targeted by one or more miRNA variants in the sRNA panel. In some embodiments, biological pathways are identified for each disease subtype, by mapping the corresponding predictive sRNA variants to targeted genes. In some embodiments, predictive isomiRs are mapped to the annotated miRNA, and the annotated miRNA used to identify potential pathways that are impacted or dysregulated by aberrant sRNA biogenesis. See Bhattacharya A, et al., PolymiRTS Database 3.0: linking polymorphisms in microRNAs and their target sites with human diseases and biological pathways, Nucleic Acids Res. 2014; 42:D86-D91.

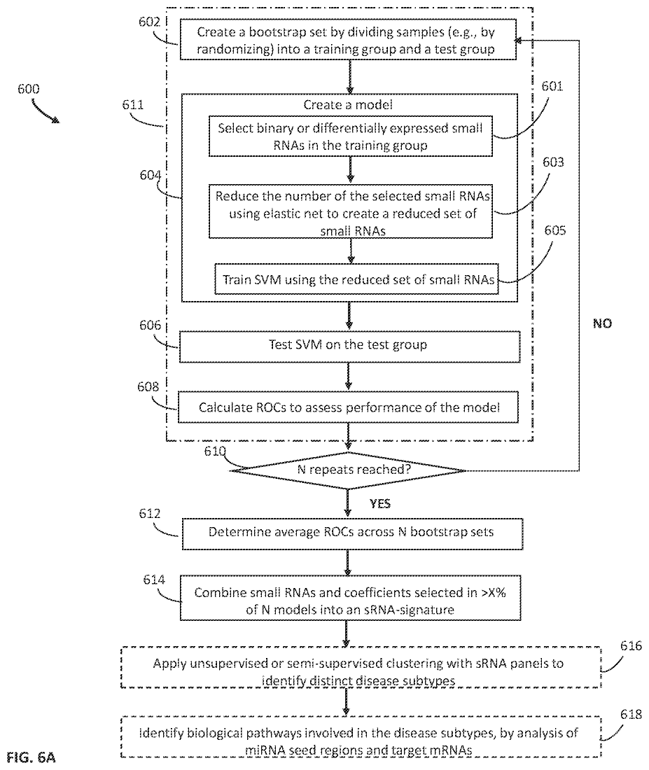

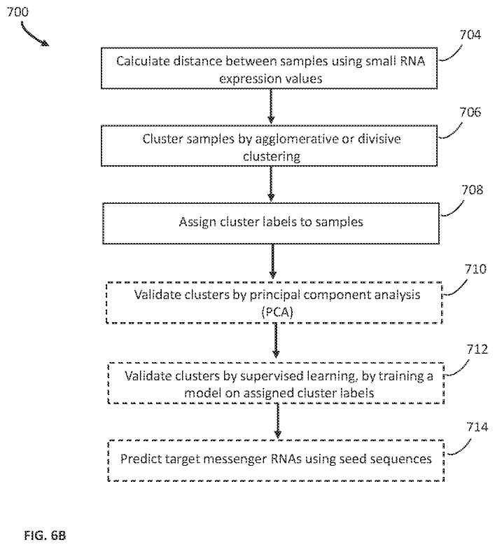

Referring to FIG. 6A, in some embodiments, the invention generates one or more sRNA panels for classifying one or more biological conditions, and for subtyping at least one of said biological conditions (e.g., in the case of a complex disease). Embodiments of FIG. 6A are illustrated in Example 3 with respect to Idiopathic Pulmonary Fibrosis (IPF).

In FIG. 6A, a process (or method) 600 can start when a plurality of samples or corresponding sRNA sequence data (adapter-trimmed as described herein) and sample metadata are acquired. A plurality of bootstrap sets can be created from the samples and analyzed, to create an sRNA signature. Referring to Block 602 of FIG. 6A, the process 600 creates a bootstrap set by dividing samples into a training group and a cross-validation or test group. The sample can be divided into a training group and a test group by randomizing, or in another manner.

To create a model, at Block 604, binary or differentially expressed sRNAs are selected in the training group (sub-block 601), and the number of candidate sRNAs are reduced, for example using elastic net (e.g., a combination of linear, logistical and ridge regression) (and as described elsewhere herein) (sub-block 603). A support vector machine (SVM) is trained using the reduced set of sRNAs, at sub-block 605. Referring to Block 606 of FIG. 6A, the SVM is tested against the test (cross-validation) group. Referring to Block 608 of FIG. 6A, Receiving Operator Characteristics (specificity, sensitivity, accuracy, etc.) are calculated to assess model performance.

As shown in FIG. 6A, the processing of the operations at Blocks 602-608 is collectively delineated as Block 611. At decision Block 610, it is determined whether the number of times (also referred to as a number of repeats) of processing at Block 611 has reached N such that the steps at Block 611 are repeated N number of times. N can be pre-selected, set based on a user input, or defined in other ways. If it is determined at decision Block 610 that the processing at Blocks 602-608 (Block 611) has been repeated N number of times (“Yes”), the process 600 continues to Block 612 where the Receiving Operator Characteristics are averaged across N number of bootstraps.

Referring to Block 614, sRNAs and coefficients selected in >X % of the N models are combined into a sRNA-signature. In some embodiments, sRNAs and coefficients selected in greater than 25% of the N models are combined into a sRNA-signature, although it is recognized that X may be a different value.

Referring to Block 616, optionally, unsupervised or semi-supervised clustering with sRNA panels (sRNAs in the signature) against samples of a biological condition (complex disease class) can be used to identify distinct disease subtypes. Referring to Block 618, optionally biological pathways involved in the disease subtypes are identified by analysis of miRNA seed regions and target mRNAs. These steps are further shown diagrammatically in FIG. 6B.

If it is determined at decision Block 610 that the processing at Blocks 602-608 (Block 611) has not been repeated N number of times (“No”), the process 600 returns to Block 602 at which another bootstrap set is created and processing at Blocks 604, 606, and 608 is repeated.

FIG. 6B illustrates a process 700 of unsupervised learning with the sRNA panels in accordance with embodiments of the present disclosure to subtype samples of a complex disease. As shown in FIG. 6B, at Block 704, the process 700 involves calculating distance between samples using the small RNA expression values. At block 706, samples are clustered by agglomerative or divisive clustering. At Block 708, cluster labels are assigned to samples. At block 710, optionally, the clusters are validated by principal component analysis. At Block 712, optionally, the clusters are validated by supervised learning (described above), by training a model on assigned cluster labels. At

Block 714, optionally target messenger RNAs are predicted using seed sequences of miRNAs in the panel used for classifying disease subtype. It should be appreciated that the order of processing at Blocks 710, 712, and 714 is shown by way of example only, as the processing at these blocks can be performed in other order(s).

In other aspects, the invention provides a method for evaluating a subject for one or more disease conditions, or disease subtype. In various embodiments, the method comprises providing a biological sample of the subject, and determining the presence or absence of sRNAs in an sRNA panel. This sRNA profile is then used to classify the condition of the subject among one or more disease conditions or disease subtypes, using a disease classifier prepared according to this disclosure.

Where the patient's condition or disease subtype is identified, the patient can be matched (i.e., administered) to the appropriate therapeutic regimen for the disease condition, and/or included or excluded from a clinical trial. For example, in some embodiments, the patient is administered a therapy that targets a dysregulated or aberrant pathway, and which corresponds to a pathway targeted by one or more sRNAs in the panel used for cluster analysis.

In various embodiments, the presence or absence, or level, of sRNAs in the subject's sample is determined by a molecular diagnostic assay, such as a quantitative PCR assay. For example, detection of the sRNA sequences is migrated to one of various detection platforms (e.g., other than RNA sequencing), which can employ reverse-transcription, amplification, and/or hybridization of a probe, including quantitative or qualitative PCR, e.g., RealTime PCR. PCR detection formats can employ stem-loop primers for RT-PCR in some embodiments, and optionally in connection with fluorescently-labeled probes.

Generally, a real-time polymerase chain reaction (qPCR) monitors the amplification of a targeted DNA molecule during the PCR, i.e. in real-time. Real-time PCR can be used quantitatively, and semi-quantitatively. Two common methods for the detection of PCR products in real-time PCR are: (1) non-specific fluorescent dyes that intercalate with any double-stranded DNA (e.g., SYBR Green (I or II)), and (2) sequence-specific DNA probes consisting of oligonucleotides that are labelled with a fluorescent reporter which permits detection only after hybridization of the probe with its complementary sequence (e.g. TAQMAN).

In some embodiments, the assay format is TAQMAN real-time PCR. TAQMAN probes are hydrolysis probes that are designed to increase the specificity of quantitative PCR. The TAQMAN probe principle relies on the 5′ to 3′ exonuclease activity of Taq polymerase to cleave a dual-labeled probe during hybridization to the complementary target sequence, with fluorophore-based detection. TAQMAN probes are dual labeled with a fluorophore and a quencher, and when the fluorophore is cleaved from the oligonucleotide probe by the Taq exonuclease activity, the fluorophore signal is detected (e.g., the signal is no longer quenched by the proximity of the labels). As in other quantitative PCR methods, the resulting fluorescence signal permits quantitative measurements of the accumulation of the product during the exponential stages of the PCR. The TAQMAN probe format provides high sensitivity and specificity of the detection.

In some embodiments, sRNAs present in the sample are converted to cDNA using specific primers, e.g., one or more stem-loop primers. Amplification of the cDNA may then be quantified in real time, for example, by detecting the signal from a fluorescent reporting molecule, where the signal intensity correlates with the level of DNA at each amplification cycle.

Alternatively, sRNAs in the panel, or their amplicons, are detected by hybridization. Exemplary platforms include surface plasmon resonance (SPR) and microarray technology. Detection platforms can use microfluidics in some embodiments, for convenient sample processing and sRNA detection.

Generally, any method for determining the presence of sRNAs in samples can be employed. Such methods further include nucleic acid sequence based amplification (NASBA), flap endonuclease-based assays, as well as direct RNA capture with branched DNA (QuantiGene™), Hybrid Capture™ (Digene), or nCounter™ miRNA detection (nanostring). The assay format, in addition to determining the presence of miRNAs and other sRNAs may also provide for the control of, inter alia, intrinsic signal intensity variation. Such controls may include, for example, controls for background signal intensity and/or sample processing, and/or hybridization efficiency, as well as other desirable controls for detecting sRNAs in patient samples (e.g., collectively referred to as “normalization controls”).

In some embodiments, the assay format is a flap endonuclease-based format, such as the Invader™ assay (Third Wave Technologies). In the case of using the invader method, an invader probe containing a sequence specific to the region 3′ to a target site, and a primary probe containing a sequence specific to the region 5′ to the target site of a template and an unrelated flap sequence, are prepared. Cleavase is then allowed to act in the presence of these probes, the target molecule, as well as a FRET probe containing a sequence complementary to the flap sequence and an auto-complementary sequence that is labeled with both a fluorescent dye and a quencher. When the primary probe hybridizes with the template, the 3′ end of the invader probe penetrates the target site, and this structure is cleaved by the Cleavase resulting in dissociation of the flap. The flap binds to the FRET probe and the fluorescent dye portion is cleaved by the Cleavase resulting in emission of fluorescence.

In some embodiments, RNA is extracted from the sample prior to sRNA processing for detection. RNA may be purified using a variety of standard procedures as described, for example, in RNA Methodologies, A laboratory guide for isolation and characterization. 2nd edition, 1998, Robert E. Farrell, Jr., Ed., Academic Press. In addition, there are various processes as well as products commercially available for isolation of small molecular weight RNAs, including mirVANA™ Paris miRNA Isolation Kit (Ambion), miRNeasy™ kits (Qiagen), MagMAX™ kits (Life Technologies), and Pure Link™ kits (Life Technologies). For example, small molecular weight RNAs may be isolated by organic extraction followed by purification on a glass fiber filter. Alternative methods for isolating miRNAs include hybridization to magnetic beads. Alternatively, miRNA processing for detection (e.g., cDNA synthesis) may be conducted in the biofluid sample, that is, without an RNA extraction step.

Generally, assays can be constructed such that each assay is at least 80%, or at least 85%, or at least 90%, or at least 95%, or at least 98% specific for the sRNA (e.g., isomiR) over an annotated sequence and/or other non-predictive iso-miRs. Annotated sequences can be determined with reference to miRBase. For example, in preparing sRNA predictor-specific real-time PCR assays, PCR primers and fluorescent probes can be prepared and tested for their level of specificity. Bicyclic nucleotides (e.g., LNA, cET, and MOE) or other nucleotide modifications (including base modifications) can be employed in probes to increase the sensitivity or specificity of detection.

In still other embodiments, sRNAs that are present in the subject's sample are determined or quantified by sRNA sequencing and adapter trimming as described elsewhere herein. sRNA sequencing can employ capture RNA sequencing, which may employ capture oligonucleotide probes to enrich/capture sRNA targets for amplification and/or sequencing. See WO 2011/06967.

As used herein, the term “about”, unless the context requires otherwise, means ±10% of an associated numerical value.

Other aspects and embodiments of the invention will be apparent from the following non-limiting examples.

### EXAMPLES

### Example 1: Construction of Multi-Class Disease Classifiers of Inflammatory Bowel Disease (IBD)

To construct disease classifiers that classify IBD samples based on the presence or absence of particular sRNA molecules, sRNA panels were determined from sequence data in various training sets representing different disease conditions of interest, such as Crohn's disease, ulcerative colitis, and diverticular disease.

Samples

All samples were collected according to their respective Institutional Review Board (IRB) approval and have patient consent for unrestricted use. Data was collected from electronic medical records and chart review. Clinical Data includes information such as: age, gender, race, ethnicity, weight, body mass index, smoking history, alcohol use history, and family history of disease. Disease-related data includes information such as: diagnosis, age at Inflammatory Bowel Disease (IBD) diagnosis, current and prior medications, comorbidities, age at proctocolectomy and Ileal Pouch Anal Anastomosis (IPAA), as well as pouch age, time from closure of ileostomy, or from pouch surgery (where applicable from patients undergoing these procedures).

Biopsies were taken from the colon epithelium. Inoperable Ulcerative Colitis (IUC), Operable Ulcerative Colitis (OUC), Crohn's Disease (CD), Diverticular Disease (DD), Polyps/Polyposis (PP), Serrated Polyps/Polyposis (SPP), colon cancer, (CC), rectal cancer (RC) were defined according to clinical, endoscopic, histologic, and imaging studies. Further inclusion criteria were the presence of ileitis for CD patients and having a normal terminal ileum as seen by endoscopy and confirmed by histology for IUC patients. Individuals who required a colonoscopy for routine screening and verified as having non-diseased bowel tissue by endoscopy and/or histology were labeled as normal controls.

All biopsies were assessed by a minimum of two (2) institutional IBD-trained pathologists and consensus scores and diagnoses were provided according to clinical and industry standard diagnostic protocols. Briefly, active inflammatory characteristics were scored according to neutrophil infiltration (0-3) and area of ulceration (0-3), each sample was classified into inactive, cryptitis, crypt abscess, numerous crypt abscesses (>3/high power field) and ulceration. Original Geboes Score (OGS) or Simplified Geboes Score (SGS) was used to classify UC. Chron's Disease Activity Index (CDAI) and Crohn's Disease Endoscopic Index of Severity (CDEIS) was used to classify CD. Hinchey Classification was used to characterize DD. Colorectal cancers, polyps and serrated polyps were classified according to the most recent recommendations of the Multi-Society Task Force on Colorectal Cancer (CRC).

An overview of the IBD samples used is displayed below:

TABLE 1

| | | Crohn's | Ulcerative | Diverticular |
|---|---|---|---|---|
| Diagnosis | Normal | disease | Colitis | Disease |
| Tissue Type | Colon | Colon | Colon | Colon |
| | Epithelium | Epithelium | Epithelium | Epithelium |
| N | 64 | 35 | 139 | 20 |
| Gender (F:M) | 26:38 | 14:21 | 50:89 | 6:14 |
| Age at sampling, years, | 56.4 ± 13.5 | 36.6 ± 15.8 | 45.5 ± 14.1 | 44.9 ± 10.6 |
| mean ± SD (range) | (26-82) | (15-76) | 32-57) | (31-69) |
| Age at IBD diagnosis, years, | NA | 30.4 ± 12.1 | 32.1 ± 11.6 | 26.2 ± 8.7 |
| mean ± SD (range) | | (18-48) | (16-51) | (21-55) |
| IBD duration, years, | NA | 13.3 | 10.5 | 12.6 |
| mean ± SD (range) | | (3-53) | (3-28) | (25-53) |
| Ashkenazi origin | 5 | 2 | 9 | 1 |
| Non-Ashkenazi origin | 53 | 31 | 120 | 17 |
| Mixed origin | 6 | 2 | 10 | 2 |
| Never smoker | 56 | 28 | 122 | 19 |
| Past smokers | 5 | 2 | 10 | 1 |
| Current smokers | 3 | 5 | 7 | 0 |
| Body mass index, | 25.5 ± 2.9 | 27.1 ± 5.3 | 25.8 ± 6.1 | 23.3 ± 5.2 |
| mean ± SD (range) | (17-30) | (18-31) | (15-41) | (18-40) |
| Family history of IBD | 2 | 3 | 8 | 1 |
| Steroid exposure | NA | NA | 110 | NA |
| Severity Score (B1:B2:B3) | NA | 7:6:8 | NA | NA |

To identify small RNA predictors for disease classes associated with IBD, small RNA sequencing data was downloaded from the GEO Database and used as a Discovery Set. Small RNA sequencing data was downloaded from the Geodatabase studies for Crohn's disease (GSE66208), Ulcerative colitis (GSE114591), Diverticular disease (GSE89667), and Normal/Control (GSE1 18504).

The data files were converted from a .sra to .fastq format using the SRA Tool Kit v2.8.0 for Centos, and .fastq-formatted files were processed as described in U.S. 2018/0258486 and International Application No. PCT/US2018/014856, filed on January 23, 2018 (which are hereby incorporated by reference in their entireties). Specifically, all .fastq data files were processed by trimming adapter sequences using the (Regex) regular expression-based search and trim algorithm, where 5′ TGGAATTCTCGGGTGCCAAGGAA 3′ (SEQ ID NO:1) (containing up to a 15 nucleotide 3′-end truncation) was input to identify the 3′ adapter sequence, and a Levenshtein Distance of 2 or a Hamming Distance of 5. Parameters for Regex searching requires that the 1^{st} nucleotide of the user-specified search term to be unaltered with respect to nucleotide insertions, deletions, and/or swaps.

To build a multi-class classifier, samples were randomized into 24 independent training and testing groups using 60% of the samples to train and 40% of the samples to test. Pre-selection chose up to 20,000 sRNAs that were present in 1 class and absent in all samples in (at least) 1 of the 3 other classes. The pre-selected sRNA had to be present at a minimum frequency of 25% in the particular class, and at least 25% in each study within the class. The sRNAs also had to be present at a minimum frequency of 25% in the test samples (e.g., all samples minus the training set). Feature reduction using an elastic net reduced the number of sRNAs to less than 126 per class, using no filter for sRNA families (such as seed sequence or non-templated 3′ additions). Testing was executed using a support vector machine with a threshold of 0.5.

Per-Class Metrics

Per-class metrics were determined for each class in order to identify markers that are most important for identifying the disease class. sRNA panels were determined from sequence data in various training sets representing different disease conditions of interest. Specific biomarker panels containing small RNA predictors of disease class were identified as follows:
Controls (Healthy individuals/“Normal” individuals): Table 2 (shows a panel of sRNA biomarkers from colon epithelium tissue for Controls (“Normal” individuals) of Inflammatory Bowel Disease);
Crohn's disease: Table 3 (shows a panel of sRNA biomarkers from colon epithelium tissue for Crohn's disease);
Ulcerative colitis: Table 4 (shows a panel of sRNA biomarkers from colon epithelium tissue for Ulcerative colitis); and
Diverticular disease: Table 5 (shows a panel of sRNA biomarkers from colon epithelium tissue for Diverticular disease).

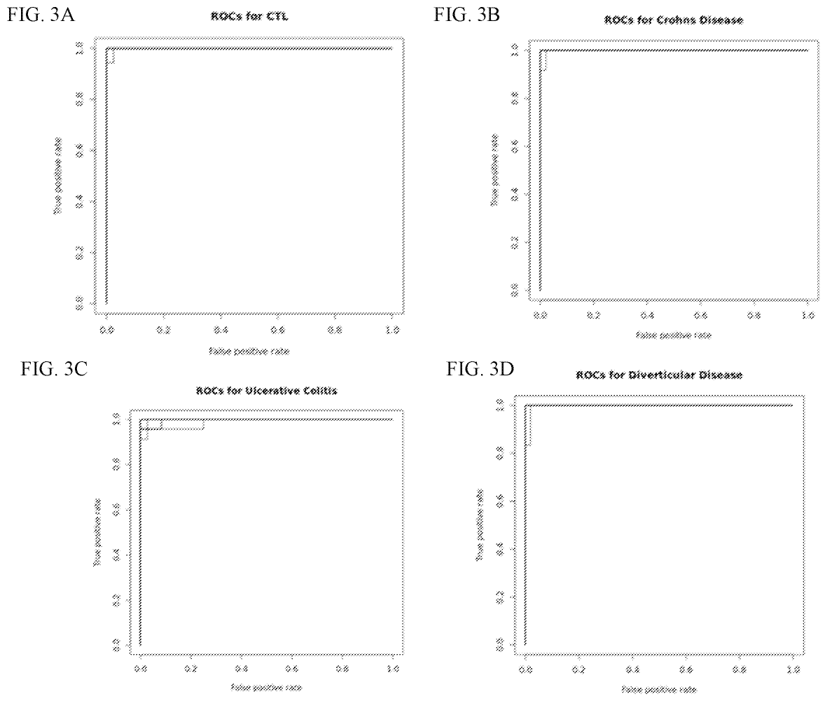

By using a supervised, non-parametric, logistical regression machine learning model, the final selection marker count was reduced from 128 to 100 maximum. In order to assess the classification model's performance, ROC/AUC curves were obtained for each set of markers identified per class, where ROC is a probability curve and AUC represents the degree or measure of separability. The ROC curve is plotted with true positive rate against the false positive rate. ROC/AUC curves were established for the various IBD classes and controls, as discussed above, and these are depicted in FIGS. 3A, 3B, 3C and 3D.

TABLE 2
Identified sRNA biomarkers in colon epithelium tissue that are associated with Normal individuals.

| SEQ | | | | | | | | | | |
|---|---|---|---|---|---|---|---|---|---|---|
| ID | | | | | | | | | | |
| NO: | Marker | importance | imp_SE | sRNA_name | ref | ext | swaps | chosen | thislbl | otherlbl |
| 2 | GCTGATTGTCACGTTCTGATT | 0.61173 | 0.11392 | hsa-mir- | (0:0) | (GC:) | (1: T > C) | 0.9 | 2.305 | 0.767 |
| | | | | 5701 | | | | | | |
| 3 | GCCCCTGGGCCTATCCTAGA | −0.50514 | 0.07172 | hsa-mir-331- | (0:−1) | (:) | ( ) | 1 | 1.473 | 2.614 |
| | | | | 3p | | | | | | |
| 4 | AGTTCTTCAGTGGCAAGCT | −0.43217 | 0.12976 | hsa-mir-22- | (0:−3) | (:) | ( ) | 0.7 | −0.639 | 0.822 |
| | | | | 5p | | | | | | |
| 5 | ACCCTGTAGAACCGAATTTGTA | 0.23477 | 0.08481 | hsa-mir-10b- | (1:−1) | (:A) | ( ) | 0.5 | 3.3 | 1.212 |
| | | | | 5p | | | | | | |
| 6 | TAGGTAGTTTCCTGTTGTTGGA | 0.17757 | 0.0569 | hsa-mir- | (0:−1) | (:AT) | (11: A > C) | 0.8 | 0.15 | −0.592 |
| | T | | | 196a-5p | | | | | | |
| 7 | ACCCTGTAGATCTGAATTTGT | 0.16483 | 0.10074 | hsa-mir-10b- | (1:−1) | (:) | (10: A > T, | 0.3 | 0.782 | −0.34 |
| | | | | 5p | | | 12: C > T) | | | |
| 8 | TGAGATGAAGCTGTAGCTC | 0.16362 | 0.03238 | hsa-mir- | (0:0) | (: C) | (8: C > A, | 0.8 | 0.779 | −0.308 |
| | | | | 4770 | | | 9: A > G) | | | |
| 9 | TACCCTGTAGAACCGAATTGGT | 0.15816 | 0.04547 | hsa-mir-10b- | (0:−1) | (:) | (19: T > G) | 0.7 | 1.483 | −0.398 |
| | | | | 5p | | | | | | |
| 10 | ACCCTGTAGAACCGAATTTGG | 0.1312 | 0.04783 | hsa-mir-10a- | (1:−2) | (:G) | (10: T > A) | 0.5 | 0.875 | −0.605 |
| | | | | 5p | | | | | | |
| 11 | TAACAGTCTACAGCCATGGTCG | −0.12465 | 0.06087 | hsa-mir-132- | (0:0) | (:) | ( ) | 0.6 | 3.56 | 4.436 |
| | | | | 3p | | | | | | |
| 12 | AGTTCTTCAGTGGCAAGCTT | −0.11012 | 0.05699 | hsa-mir-22- | (0:−2) | (:) | ( ) | 0.3 | −0.394 | 1.187 |
| | | | | 5p | | | | | | |
| 13 | TACCCTGTAGAACCGAATTTGG | 0.09977 | 0.03596 | hsa-mir-10b- | (0:−2) | (:G) | ( ) | 0.5 | 4.121 | 1.664 |
| | | | | 5p | | | | | | |
| 14 | CAGTGCAATGATGAAAGGGCAT | −0.08933 | 0.05037 | hsa-mir- | (0:0) | (:) | (10: T > A, | 0.3 | 0.717 | 2.623 |
| | | | | 130a-3p | | | 12: A > G) | | | |
| 15 | TACCCTGTAGAACCGAATTTA | 0.07544 | 0.04788 | hsa-mir-10b- | (0:−3) | (:A) | ( ) | 0.4 | 2.698 | 0.845 |
| | | | | 5p | | | | | | |
| 16 | TACAGTTGTTCAACCAGTTACT | −0.07464 | 0.05019 | hsa-mir-582- | (1:0) | (:) | ( ) | 0.2 | −0.358 | 0.671 |
| | | | | 5p | | | | | | |
| 17 | ACCCTGTAGAACCGAATTTGGG | 0.06375 | 0.06375 | hsa-mir-10a- | (1:0) | (:) | (10: T > A, | 0.1 | 0.747 | −0.188 |
| | | | | 5p | | | 20: T > G) | | | |
| 18 | TACCCTGTAGGACCGAATTTGT | 0.05883 | 0.03032 | hsa-mir-10b- | (0:−1) | (:) | (10: A > G) | 0.4 | 1.962 | −0.355 |
| | | | | 5p | | | | | | |
| 19 | TGGCAGTGTCTTAGCTGGTT | −0.05794 | 0.04762 | hsa-mir-34a- | (0:−2) | (:) | ( ) | 0.2 | −0.482 | 1.044 |
| | | | | 5p | | | | | | |
| 20 | ACCCTGTAGAACCGAATTTA | 0.04848 | 0.03233 | hsa-mir-10a- | (1:−3) | (:A) | (10: T > A) | 0.2 | 0.32 | −0.63 |
| | | | | 5p | | | | | | |
| 21 | ACCCTGTAGAACCGAATTTGTT | 0.04605 | 0.04605 | hsa-mir-10b- | (1:−1) | (:T) | ( ) | 0.1 | 1.076 | −0.146 |
| | | | | 5p | | | | | | |
| 22 | TACCCTGTAGATCCGATTTTGT | 0.04078 | 0.01861 | hsa-mir-10b- | (0:−1) | (:) | (11: A > T, | 0.4 | 1.192 | −0.283 |
| | | | | 5p | | | 16: A > T) | | | |
| 23 | TACCCTGTAGAACCGAGTTTGT | 0.03972 | 0.03306 | hsa-mir-10b- | (0:−1) | (:) | (16: A > G) | 0.2 | 2.752 | 0.399 |
| | | | | 5p | | | | | | |
| 24 | TTCAAGTAATCCAGGATAGGCC | 0.03965 | 0.03658 | hsa-mir-26a- | (0:−1) | (:CT) | ( ) | 0.2 | 0.841 | −0.548 |
| | T | | | 5p | | | | | | |
| 25 | TACCCTGTAGAACCGAATTTAT | 0.03939 | 0.03051 | hsa-mir-10b- | (0:−1) | (:) | (20: G > A) | 0.2 | 1.886 | 0.183 |
| | | | | 5p | | | | | | |
| 26 | TACCCTGTAGAACCGGATTTG | 0.03714 | 0.02781 | hsa-mir-10b- | (0:−2) | (:) | (15: A > G) | 0.2 | 0.166 | −0.663 |
| | | | | 5p | | | | | | |
| 27 | TATTGCACTTGTCCCGGCCTGT | 0.03206 | 0.03206 | hsa-mir-92a- | (0:2) | (:C) | (22: G > A) | 0.1 | 0.533 | −0.546 |
| | AGC | | | 3p | | | | | | |
| 28 | ACCCTGTAGATCTGAATTTGTG | 0.02789 | 0.02789 | hsa-mir-10a- | (1:0) | (:A) | (12: C > T) | 0.1 | 0.267 | −0.681 |
| | A | | | 5p | | | | | | |
| 29 | CACTAGATTGTGAGCTCCT | 0.02652 | 0.02652 | hsa-mir-28- | (0:−3) | (:) | ( ) | 0.1 | 2.028 | 0.439 |
| | | | | 3p | | | | | | |
| 30 | TACCCTGTAGTACCGAATTTGT | 0.02641 | 0.02641 | hsa-mir-10b- | (0:−1) | (:) | (10: A > T) | 0.1 | 1.227 | −0.21 |
| | | | | 5p | | | | | | |
| 31 | CAGTGCAATGTTAAAAGGGCAA | −0.026 | 0.01733 | hsa-mir- | (0:−1) | (:A) | (10: A > T, | 0.2 | −0.212 | 1.183 |
| | | | | 130b-3p | | | 12: G > A) | | | |
| 32 | CTGACCTATGATTTGACAGCC | 0.02413 | 0.01324 | hsa-mir-192- | (0:0) | (:) | (11: A > T) | 0.3 | 1.746 | 0.096 |
| | | | | 5p | | | | | | |
| 33 | CTGACCTATGAATTGACAGCCC | 0.02306 | 0.01562 | hsa-mir-192- | (0:0) | (:CT) | ( ) | 0.2 | 2.004 | 0.427 |
| | T | | | 5p | | | | | | |
| 34 | CCACTGCCCCAGGTGCTGCTGG | −0.02248 | 0.02248 | hsa-mir-324- | (−2:0) | (:) | ( ) | 0.1 | −0.481 | 0.945 |
| | | | | 3p | | | | | | |
| 35 | TGAGGTAGTAGGTTGTGTGGGT | 0.02215 | 0.02215 | hsa-let-7c- | (0:0) | (:) | (16: A > G, | 0.1 | 0.975 | −0.325 |
| | | | | 5p | | | 20: T > G) | | | |
| 36 | ACTGTGCGTGTGACAGCGGCT | −0.02097 | 0.01562 | hsa-mir-210- | (−1:−2) | (:) | ( ) | 0.2 | −0.666 | 0.215 |
| | | | | 3p | | | | | | |
| 37 | CTGCGCAAGCTACTGCCTTG | −0.0202 | 0.0202 | hsa-let-7i-3p | (0:−2) | (:) | ( ) | 0.1 | 1.199 | 2.896 |
| 38 | CACCCGTAGAACCGACCTTGCG | −0.02011 | 0.01097 | hsa-mir-99b- | (0:0) | (:A) | ( ) | 0.3 | 3.612 | 4.648 |
| | A | | | 5p | | | | | | |
| 39 | CTGACCTATGTATTGACAGCC | 0.01839 | 0.01249 | hsa-mir-192- | (0:0) | (:) | (10: A > T) | 0.2 | 2.279 | 0.663 |
| | | | | 5p | | | | | | |
| 40 | TACCCTGTAGAACCGAATTTGC | 0.01577 | 0.01577 | hsa-mir-10b- | (0:−2) | (:C) | ( ) | 0.1 | 4.555 | 1.079 |
| | | | | 5p | | | | | | |
| 41 | TGAGAACTGAATTCCATAGGCT | −0.01551 | 0.01551 | hsa-mir- | (0:1) | (:AA) | (17: G > A, | 0.1 | −0.359 | 0.464 |
| | GAA | | | 146a-5p | | | 20: T > C) | | | |
| 42 | TGACCTATGAATTGACAGCCAA | 0.01402 | 0.01402 | hsa-mir-215- | (1:3) | (:T) | (18: A > C) | 0.1 | 0.754 | −0.46 |
| | TT | | | 5p | | | | | | |
| 43 | TACCCTGTAGAACCGAATTTGT | 0.01382 | 0.01382 | hsa-mir-10b- | (0:−1) | (:A) | ( ) | 0.1 | 5.669 | 4.122 |
| | A | | | 5p | | | | | | |
| 44 | TGAGATGAAGCACTGTAGATC | 0.01158 | 0.01158 | hsa-mir-143- | (0:0) | (:) | (18: C > A) | 0.1 | 2.526 | 1.048 |
| | | | | 3p | | | | | | |
| 45 | TACCCTGTAGAACCGAACTTGT | 0.0115 | 0.00939 | hsa-mir-10b- | (0:−1) | (:) | (17: T > C) | 0.2 | 1.946 | 0.086 |
| | | | | 5p | | | | | | |
| 46 | CTGACCTATGAACTGACAGCC | 0.01068 | 0.0088 | hsa-mir-192- | (0:0) | (:) | (12: T > C) | 0.2 | 2.713 | 0.568 |
| | | | | 5p | | | | | | |
| 47 | GATTGTCACGTTCTGATT | 0.00994 | 0.00994 | hsa-mir- | (2:0) | (G:) | ( ) | 0.1 | 0.926 | −0.013 |
| | | | | 5701 | | | | | | |
| 48 | TTACAGTCTACAGCCATGGTCG | −0.007 | 0.007 | hsa-mir-132- | (0:0) | (:) | (1: A > T) | 0.1 | −0.541 | 0.325 |
| | | | | 3p | | | | | | |
| 49 | CATTGCACTTGTCTCGGTCTGA | 0.00642 | 0.00642 | hsa-mir-25- | (0:0) | (:AT) | ( ) | 0.1 | 2.02 | 0.798 |
| | AT | | | 3p | | | | | | |
| 50 | TACCCTGTTGAACCGAATTTGT | 0.00629 | 0.00629 | hsa-mir-10b- | (0:−1) | (:) | (8: A > T) | 0.1 | 0.959 | −0.227 |
| | | | | 5p | | | | | | |
| 51 | CAAAGTGCTGTTCGTGCAGGTA | −0.00623 | 0.00623 | hsa-mir-93- | (0:−1) | (:) | ( ) | 0.1 | 2.94 | 3.614 |
| | | | | 5p | | | | | | |
| 52 | CTCGCTTCTGGCGCCAAGCGCC | −0.00413 | 0.00413 | <NA> | (NA:NA) | (NA:NA) | ( ) | 0.1 | −0.552 | 0.651 |
| | CGGC | | | | | | | | | |
| 53 | AACTGGCCCTCAAAGTCCCG | −0.00368 | 0.00368 | hsa-mir- | (0:−2) | (:) | ( ) | 0.1 | 0.083 | 1.702 |
| | | | | 193b-3p | | | | | | |
| 54 | TGAGAACTGAATTCCATAGGCA | −0.00364 | 0.00364 | hsa-mir- | (0:−1) | (:AA) | ( ) | 0.1 | 0.256 | 1.187 |
| | A | | | 146b-5p | | | | | | |
| 55 | TGAGGTAGTAGATTGTATAGTT | 0.00325 | 0.00325 | hsa-let-7a- | (0:2) | (:) | (11: G > A) | 0.1 | 0.75 | −0.212 |
| | TT | | | 5p | | | | | | |
| 56 | ACCCTGTAGATCCGAAT | 0.00148 | 0.00148 | hsa-mir-10a- | (1:−5) | (:) | ( ) | 0.1 | 0.215 | −0.459 |
| | | | | 5p | | | | | | |
| 57 | AGGCTGTGATGCTCTCCTGAGC | 0.00039 | 0.00039 | hsa-mir- | (0:−1) | (:CT) | ( ) | 0.1 | 0.595 | −0.142 |
| | CCT | | | 7974 | | | | | | |
| 58 | TAACACTGTCTGGTAAC | 0.00027 | 0.00027 | hsa-mir- | (0:−5) | (:) | ( ) | 0.1 | 1.631 | −0.336 |
| | | | | 200a-3p | | | | | | |
| 59 | TACCCTGTAGATCCGAATTCGT | 0.00024 | 0.00024 | hsa-mir-10b- | (0:−1) | (:) | (11: A > T, | 0.1 | 1.832 | −0.081 |
| | | | | 5p | | | 19: T > C) | | | |

TABLE 3
Identified sRNA biomarkers in colon epithelium tissue that are associated with Crohn's disease.

| SEQ ID | | | | | | | | | | |
|---|---|---|---|---|---|---|---|---|---|---|
| NO: | Marker | importance | imp_SE | sRNA_name | ref | ext | swaps | chosen | thislbl | otherlbl |
| 60 | CCGCCCCACCCCGCGCGCGCCGC | 0.74618 | 0.16463 | <NA> | (NA:NA) | (NA:NA) | ( ) | 0.8 | 1.72 | −0.59 |
| 61 | CGCTTCTGGCGCCAAGCGCCCGGC | 0.25545 | 0.08406 | <NA> | (NA:NA) | (NA:NA) | ( ) | 0.7 | 1.39 | −0.62 |
| | CGC | | | | | | | | | |
| 62 | AGATTGAGGGTTCGTCCCTTCGTG | 0.25408 | 0.05563 | <NA> | (NA:NA) | (NA:NA) | ( ) | 0.8 | 2.73 | −0.37 |
| | GTCGCC | | | | | | | | | |
| 63 | GGCTTGGTCTAGGGGTATGATTCT | 0.21881 | 0.06902 | <NA> | (NA:NA) | (NA: NA) | ( ) | 0.7 | 2.2 | −0.46 |
| | CGCTTT | | | | | | | | | |
| 64 | GGCTTTGTCTAGGGGTATGATTCT | 0.18401 | 0.12882 | <NA> | (NA:NA) | (NA:NA) | ( ) | 0.4 | 1.34 | −0.65 |
| | CGCTT | | | | | | | | | |
| 65 | CCCGCCCCACCCCGCGCGCGCCGC | 0.15615 | 0.09596 | <NA> | (NA:NA) | (NA:NA) | ( ) | 0.3 | 1.5 | −0.64 |
| | T | | | | | | | | | |
| 66 | CGTACGGAAGACCCGCTCCCCGGC | 0.11296 | 0.05941 | <NA> | (NA:NA) | (NA:NA) | ( ) | 0.3 | 1.26 | −0.61 |
| | GCCGCT | | | | | | | | | |
| 67 | GTACGGAAGACCCGCTCCCCGGCG | 0.10944 | 0.10944 | <NA> | (NA:NA) | (NA:NA) | ( ) | 0.1 | 1.36 | −0.59 |
| | CCG | | | | | | | | | |
| 68 | TGGTCTAGCGGTTAGGATTCCTGG | 0.09687 | 0.06389 | <NA> | (NA:NA) | (NA:NA) | ( ) | 0.3 | 1.02 | −0.66 |
| | TTTT | | | | | | | | | |
| 69 | CGCCCCACCCCGCGCGCGCCGC | 0.09422 | 0.03815 | <NA> | (NA:NA) | (NA:NA) | ( ) | 0.5 | 1.64 | −0.61 |
| 70 | CCCGCGAGGGGGGCCCGGGCAC | 0.07217 | 0.05546 | <NA> | (NA:NA) | (NA:NA) | ( ) | 0.2 | 1.03 | −0.58 |
| 71 | GCGCCGCCGCCCCCCCCACGCCCG | 0.06871 | 0.04611 | <NA> | (NA:NA) | (NA:NA) | ( ) | 0.2 | 1.64 | −0.67 |
| | GGGC | | | | | | | | | |
| 72 | GCTCCCCGTCCTCCCCCCTCCCC | 0.06762 | 0.06762 | <NA> | (NA:NA) | (NA:NA) | ( ) | 0.1 | 1.58 | −0.67 |
| 73 | GCGCAATGAAGGTGAAGGCCGGC | 0.06288 | 0.03999 | <NA> | (NA:NA) | (NA:NA) | ( ) | 0.4 | 1.03 | −0.6 |
| | GC | | | | | | | | | |
| 74 | ACGCTGCCAGTTGAAGAACTGT | 0.05063 | 0.05063 | hsa-mir-22- | (0:0) | (:) | (1: A > C) | 0.1 | 0.86 | −0.46 |
| | | | | 3p | | | | | | |
| 75 | GCCCCTGGGCCTATCCTAGAAAA | 0.04958 | 0.03308 | hsa-mir-331- | (0:0) | (:AA) | ( ) | 0.2 | 0.68 | −0.65 |
| | | | | 3p | | | | | | |
| 76 | GCGGGTCCGGCCGTGTCGGCGGC | 0.04831 | 0.04831 | <NA> | (NA:NA) | (NA:NA) | ( ) | 0.1 | 0.65 | −0.67 |
| 77 | GGCTTGGTCTAGGGGTATGATTCT | 0.04437 | 0.04437 | <NA> | (NA:NA) | (NA:NA) | ( ) | 0.1 | 3.5 | 0.65 |
| | CGCT | | | | | | | | | |
| 78 | CCACCTCCCCTGCAAACGTCC | 0.03994 | 0.02586 | hsa-mir- | (0:−1) | (:) | ( ) | 0.4 | 0.46 | −0.6 |
| | | | | 1306-5p | | | | | | |
| 79 | GGTTAGGATTCCTGGTTTT | 0.03829 | 0.03829 | <NA> | (NA:NA) | (NA:NA) | ( ) | 0.1 | 1.08 | −0.57 |
| 80 | TCTGGCATGCTAACTAGTTACGCG | 0.03622 | 0.03622 | <NA> | (NA:NA) | (NA:NA) | ( ) | 0.1 | 0.84 | −0.67 |
| | ACCCCC | | | | | | | | | |
| 81 | CGCGTCCCCCGAAGAGGGGGACG | 0.03391 | 0.03391 | <NA> | (NA:NA) | (NA:NA) | ( ) | 0.1 | 1.08 | −0.68 |
| | GCGGAGC | | | | | | | | | |
| 82 | GCGGAGCGAGCGCACGGGGTCGG | 0.0323 | 0.0323 | <NA> | (NA:NA) | (NA:NA) | ( ) | 0.1 | 0.79 | −0.52 |
| | CGGCGAC | | | | | | | | | |
| 83 | CCCCCGCCCCACCCCGCGCGCGCC | 0.02563 | 0.02563 | <NA> | (NA:NA) | (NA:NA) | ( ) | 0.1 | 1.3 | −0.68 |
| | GCTCGC | | | | | | | | | |
| 84 | CCGTAGGTGAACCTGCGGAAGGAT | 0.02433 | 0.01963 | <NA> | (NA:NA) | (NA:NA) | ( ) | 0.2 | 2.36 | −0.5 |
| | CATTA | | | | | | | | | |
| 85 | GGGCTACGCCTGTCTGAGCGTCGC | 0.02206 | 0.02206 | <NA> | (NA:NA) | (NA:NA) | ( ) | 0.1 | 2.74 | 0.07 |
| | TT | | | | | | | | | |
| 86 | GCTACGCCTGTCTGAGCGTCGCTT | 0.02103 | 0.02103 | <NA> | (NA:NA) | (NA:NA) | ( ) | 0.1 | 1.48 | −0.46 |
| 87 | CCCCCACAACCGCGCTTGACTAGCT | 0.0204 | 0.0204 | <NA> | (NA:NA) | (NA:NA) | ( ) | 0.1 | 1.43 | −0.36 |
| | T | | | | | | | | | |
| 88 | CCCTACCCCCCCGGCCCCGTC | 0.01307 | 0.01307 | <NA> | (NA:NA) | (NA:NA) | ( ) | 0.1 | 1.25 | −0.56 |
| 89 | CCCGCCCCACCCCGCGCGCGCCGC | 0.01108 | 0.01108 | <NA> | (NA:NA) | (NA:NA) | ( ) | 0.1 | 1.7 | −0.59 |
| | TCGC | | | | | | | | | |
| 90 | GGGGGTATAGCTCAGTGGTAGAG | 0.01022 | 0.01022 | <NA> | (NA:NA) | (NA:NA) | ( ) | 0.1 | 1.12 | −0.58 |
| | CGTGCTT | | | | | | | | | |
| 91 | GTCGGTCGGGCTGGGGCGCGAAG | 0.00996 | 0.00996 | <NA> | (NA:NA) | (NA:NA) | ( ) | 0.1 | 2.53 | −0.51 |
| | CGGGGCT | | | | | | | | | |
| 92 | TCAGTGGAGAGCATTTGACT | 0.00991 | 0.00991 | <NA> | (NA:NA) | (NA:NA) | ( ) | 0.1 | 0.54 | −0.66 |
| 93 | CACCCCTAGAACCGACCTTGCG | 0.0095 | 0.0095 | hsa-mir-99b- | (0:0) | (:) | (5: G > C) | 0.1 | 0.17 | −0.66 |
| | | | | 5p | | | | | | |
| 94 | CCTCACCATCCCTTCTGCCTGCA | 0.00892 | 0.00892 | hsa-mir- | (0:1) | (:) | ( ) | 0.1 | 0.2 | −0.65 |
| | | | | 6511a-3p | | | | | | |
| 95 | GTCAGGATGGCCGAGCGGTCT | 0.00647 | 0.00647 | <NA> | (NA:NA) | (NA:NA) | ( ) | 0.1 | 2.13 | 0.36 |
| 96 | TCCCTGGTCTAGTGGTTAGGATTC | 0.00644 | 0.00644 | <NA> | (NA:NA) | (NA:NA) | ( ) | 0.1 | 1.6 | −0.27 |
| | GGCGCG | | | | | | | | | |
| 97 | TGAGATGAAGCACTGTAGATC | −0.00555 | 0.00555 | hsa-mir-143- | (0:0) | (:) | (18: C > A) | 0.1 | −0.07 | 1.91 |
| | | | | 3p | | | | | | |
| 98 | GGATCGGCCCCGCCGGGGTCGGC | 0.00523 | 0.00523 | <NA> | (NA:NA) | (NA:NA) | ( ) | 0.1 | 1.04 | −0.68 |
| 99 | GGAACCTGCGGAAGGATCATTA | 0.00215 | 0.00215 | <NA> | (NA:NA) | (NA:NA) | ( ) | 0.1 | 2.24 | −0.33 |
| 100 | TGAGGTAGTAGGTTGTATGGTTG | 0.00179 | 0.00179 | hsa-mir-4510 | (0:1) | (:) | (5: G > T, | 0.1 | 0.92 | −0.53 |
| | | | | | | | 12: A > T) | | | |
| 101 | GTCTAGTGGTTAGGATTCGGCGCT | 0.00093 | 0.00093 | <NA> | (NA:NA) | (NA:NA) | ( ) | 0.1 | 1.61 | −0.38 |
| 102 | TCCCTGGTCTAGTGGCTAGGATTC | 0.00085 | 0.00085 | <NA> | (NA:NA) | (NA:NA) | ( ) | 0.1 | 0.72 | −0.64 |
| | GGCGCT | | | | | | | | | |
| 103 | GCCGCCCCCCCCACGCCCGGGGC | 0.0002 | 0.0002 | <NA> | (NA:NA) | (NA:NA) | ( ) | 0.1 | 0.59 | −0.68 |

TABLE 4
Identified sRNA biomarkers in colon epithelium tissue that are associated with Ulcerative colitis.

| SEQ ID | | | | | | | | | | |
|---|---|---|---|---|---|---|---|---|---|---|
| NO: | Marker | importance | imp_SE | sRNA_name | ref | ext | swaps | chosen | thislbl | otherlbl |
| 104 | TGTCAGTTTGTCAAATACCC | 0.46706 | 0.1009 | hsa-mir-223- | (0:2) | (:) | ( ) | 0.9 | 1.892 | 0.1084 |
| | CAAG | | | 3p | | | | | | |
| 105 | CAGCAGCAATTCATGTTTTG | 0.29749 | 0.09883 | hsa-mir-424- | (0:0) | (:T) | ( ) | 0.6 | 0.578 | −0.613 |
| | AAT | | | 5p | | | | | | |
| 106 | GTGGTTGTAGTCCGTGCGA | −0.22154 | 0.09667 | <NA> | (NA:NA) | (NA:NA) | ( ) | 0.5 | −0.373 | 1.2368 |
| | GAATACC | | | | | | | | | |
| 107 | GGATATCATCATATACTGTA | 0.1973 | 0.11602 | hsa-mir-144- | (0:1) | (:) | ( ) | 0.4 | 2.428 | 0.8535 |
| | AGT | | | 5p | | | | | | |
| 108 | TAACAGTCTCCAGTCACGG | 0.14329 | 0.07797 | hsa-mir-212- | (0:−1) | (:) | ( ) | 0.6 | 1.215 | −0.5329 |
| | C | | | 3p | | | | | | |
| 109 | TCAGTGCACTACAGAACTTT | 0.13604 | 0.06626 | hsa-mir- | (0:0) | (:T) | (20: G > T) | 0.5 | 0.643 | −0.6209 |
| | TTT | | | 148a-3p | | | | | | |
| 110 | CCAGTGGGGCTGCTGTTAT | −0.13318 | 0.07284 | hsa-mir-194- | (0:0) | (:T) | ( ) | 0.3 | 0.857 | 2.7111 |
| | CTGT | | | 3p | | | | | | |
| 111 | GATAAAGTAGAAAGCACTA | 0.13252 | 0.06175 | hsa-mir-142- | (1:0) | (G:) | ( ) | 0.4 | 1.653 | −0.6021 |
| | CT | | | 5p | | | | | | |
| 112 | TAGGTAGTTTCCTGTTGTTG | −0.1183 | 0.04091 | hsa-mir- | (0:−1) | (:AT) | (11: A > C) | 0.6 | −0.676 | −0.1724 |
| | GAT | | | 196a-5p | | | | | | |
| 113 | ATGCTTATCAGACTGATGTT | 0.11425 | 0.07239 | hsa-mir-21- | (2:0) | (AT:) | ( ) | 0.3 | 1.241 | −0.512 |
| | GA | | | 5p | | | | | | |
| 114 | TAGTGCAATATTGCTTATAG | 0.10893 | 0.0759 | hsa-mir-454- | (0:−1) | (:) | ( ) | 0.3 | 0.82 | 0.0483 |
| | GG | | | 3p | | | | | | |
| 115 | CCCATAAAGTAGAAAGCAC | 0.10582 | 0.05342 | hsa-mir-142- | (−2:0) | (:) | ( ) | 0.5 | 1.414 | −0.294 |
| | TACT | | | 5p | | | | | | |
| 116 | TACCCATTGCATATCGGAGT | 0.097 | 0.07557 | hsa-mir-660- | (0:−1) | (:) | ( ) | 0.3 | 0.876 | −0.4505 |
| | T | | | 5p | | | | | | |
| 117 | ACTGGACTTGGAGTCAGAA | −0.09333 | 0.05017 | hsa-mir- | (0:3) | (:A) | (13: G > T, | 0.3 | 2.232 | 4.1887 |
| | GGAA | | | 378b | | | 19: A > G) | | | |
| 118 | AAGCAGCAATTCATGTTTTG | 0.09165 | 0.06219 | hsa-mir-424- | (1:−1) | (A:) | ( ) | 0.2 | 0.263 | −0.6458 |
| | A | | | 5p | | | | | | |
| 119 | CTGCAGCACGTAAATATTG | 0.0866 | 0.05794 | hsa-mir-16- | (2:0) | (CT:) | ( ) | 0.2 | 0.882 | −0.5753 |
| | GCG | | | 5p | | | | | | |
| 120 | TGGCAGTGTCTTAGCTGGT | 0.07815 | 0.06409 | hsa-mir-34a- | (0:−2) | (:) | ( ) | 0.3 | 1.71 | −0.1242 |
| | T | | | 5p | | | | | | |
| 121 | ACTGGACTTGGAGTCAGAA | −0.07752 | 0.052 | hsa-mir- | (0:−2) | (:) | (20: A > G, | 0.2 | −0.284 | 1.3769 |
| | GGTT | | | 378c | | | 21: G > T) | | | |
| 122 | TGAGAACTGAATTCCATAG | 0.07149 | 0.03423 | hsa-mir- | (0:4) | (:) | (24: G > A) | 0.6 | 2.372 | 0.6917 |
| | GCTGTAA | | | 146b-5p | | | | | | |
| 123 | ACTGGACTTGGAGTCAGAA | −0.0679 | 0.04539 | hsa-mir- | (0:−2) | (:) | (20: A > G, | 0.2 | 0.289 | 2.0819 |
| | GGAT | | | 378c | | | 21: G > A) | | | |
| 124 | TGAGAACTGAATTCCATAG | 0.06566 | 0.04343 | hsa-mir- | (0:4) | (:T) | (24: G > A) | 0.3 | 0.687 | −0.4488 |
| | GCTGTAAT | | | 146b-5p | | | | | | |
| 125 | GTTGAGACTCTGAAATCTG | −0.06461 | 0.05023 | hsa-mir- | (−2:−7) | (G:GATT) | (3: C > G, | 0.2 | −0.649 | 0.1771 |
| | ATT | | | 4431 | | | 14: A > A) | | | |
| 126 | TTAATGCTAATCGTGATAG | 0.06346 | 0.02758 | hsa-mir-155- | (0:−4) | (:) | ( ) | 0.4 | 2.46 | 0.3365 |
| | | | | 5p | | | | | | |
| 127 | TGAGAACTGAATTCCATAG | 0.06095 | 0.0468 | hsa-mir- | (0:−2) | (:AA) | (17: G > A) | 0.2 | 1.103 | 0.1217 |
| | GAA | | | 146a-5p | | | | | | |
| 128 | CTATACGACCTGCTGCCTTT | −0.05799 | 0.05799 | hsa-let-7d- | (0:−1) | (:A) | ( ) | 0.1 | 0.725 | 1.845 |
| | CA | | | 3p | | | | | | |
| 129 | TACCCTGTAGAACCGAATTT | −0.05773 | 0.04012 | hsa-mir-lOa- | (0:0) | (:) | (11: T > A, | 0.2 | −0.445 | 0.5034 |
| | GCG | | | 5p | | | 21: T > C) | | | |
| 130 | TGGCAGTGTCTTAGCTGGT | 0.05695 | 0.04073 | hsa-mir-34a- | (0:−3) | (:) | ( ) | 0.2 | 0.721 | −0.5822 |
| | | | | 5p | | | | | | |
| 131 | CCAGTGGGGCTGCTGTTAT | −0.05534 | 0.03762 | hsa-mir-194- | (0:−1) | (:) | ( ) | 0.3 | 1.163 | 2.2638 |
| | CT | | | 3p | | | | | | |
| 132 | TTGAGAACTGAATTCCATG | 0.05453 | 0.04544 | hsa-mir- | (−1:0) | (:) | ( ) | 0.2 | 2.563 | 0.8429 |
| | GGTT | | | 146a-5p | | | | | | |
| 133 | TTACAGTCTACAGCCATGGT | 0.04999 | 0.04437 | hsa-mir-132- | (0:0) | (:) | (1: A > T) | 0.2 | 0.833 | −0.4181 |
| | CG | | | 3p | | | | | | |
| 134 | ACTGGACTTGGAGTCAGAA | −0.04834 | 0.0324 | hsa-mir- | (0:3) | (:) | (19: A > G, | 0.2 | 5.356 | 6.5699 |
| | GGCT | | | 378d | | | 20: A > G) | | | |
| 135 | TGAGAACTGAATTCCATAG | 0.04829 | 0.0337 | hsa-mir- | (0:2) | (:AG) | ( ) | 0.2 | 0.761 | −0.2346 |
| | GCTGTAG | | | 146b-5p | | | | | | |
| 136 | CCCATAAAGTAGAAAGCAC | 0.04703 | 0.03279 | hsa-mir-142- | (−2:−1) | (:A) | ( ) | 0.2 | 2.327 | 0.2258 |
| | TACA | | | 5p | | | | | | |
| 137 | TGAGGTAGTAGTTTGTGCT | 0.04637 | 0.04637 | hsa-let-7i-5p | (0:−3) | (:) | ( ) | 0.1 | 3.668 | 2.5754 |
| 138 | CGGCGCAAGCTACTGCCTT | 0.04625 | 0.04625 | hsa-let-7i-3p | (0:−2) | (:) | (1: T > G) | 0.1 | 0.127 | −0.6692 |
| | G | | | | | | | | | |
| 139 | AGTTCTTCAGTGGCAAGCT | 0.04577 | 0.04577 | hsa-mir-22- | (0:−3) | (:) | ( ) | 0.1 | 1.084 | −0.0644 |
| | | | | 5p | | | | | | |
| 140 | TCCCCTGTAGAACCGAATTT | −0.04267 | 0.02897 | hsa-mir- | (0:−1) | (:) | (1: A > C) | 0.2 | −0.655 | 0.1801 |
| | GT | | | 10b-5p | | | | | | |
| 141 | ACTGGACTTGGAGTCAGAA | −0.04209 | 0.02716 | hsa-mir- | (0:0) | (:ATT) | (9: A > G, | 0.3 | 1.615 | 3.1346 |
| | GGCATT | | | 422a | | | 11: G > A) | | | |
| 142 | AAGCTCGGTCTGAGGCCCC | −0.04032 | 0.03266 | hsa-mir-423- | (−1:−2) | (:) | ( ) | 0.2 | 0.598 | 1.7929 |
| | TCA | | | 3p | | | | | | |
| 143 | CCAGTGGGGCTGCTGTTAT | −0.03971 | 0.03971 | hsa-mir-194- | (0:0) | (:A) | ( ) | 0.1 | −0.383 | 1.5327 |
| | CTGA | | | 3p | | | | | | |
| 144 | TGAGGGAGTAGTTTGTGCT | 0.03743 | 0.02474 | hsa-let-7i-5p | (0:0) | (:A) | (5: T > G) | 0.3 | 0.516 | −0.5159 |
| | GTTA | | | | | | | | | |
| 145 | AAGAAAGTAGAAAGCACTA | 0.03726 | 0.03726 | hsa-mir-142- | (1:0) | (A:) | (1: T > A) | 0.1 | 0.759 | −0.6659 |
| | CT | | | 5p | | | | | | |
| 146 | CGCTGCCAGTTGAAGAACT | 0.03671 | 0.03671 | hsa-mir-22- | (2:0) | (C:) | ( ) | 0.1 | 1.055 | −0.5449 |
| | GT | | | 3p | | | | | | |
| 147 | GGCTGGTCCGATGGTAGT | −0.03534 | 0.03534 | hsa-mir- | (0:−1) | (:) | (8: A > C, | 0.1 | 0.079 | 1.378 |
| | | | | 6131 | | | 14: G > T) | | | |
| 148 | CTGGGAGAAGGCTGTTTAC | −0.03467 | 0.03467 | hsa-mir-30c- | (0:0) | (:) | ( ) | 0.1 | 0.783 | 1.6525 |
| | TCT | | | 2-3p | | | | | | |
| 149 | AAGCAATTCTCAAAGGAGC | 0.03329 | 0.01693 | hsa-mir- | (−3:−5) | (:) | ( ) | 0.4 | 0.38 | −0.6931 |
| | | | | 5571-5p | | | | | | |
| 150 | CTCGGCGCCCCCTCGATGCT | −0.03132 | 0.02602 | <NA> | (NA:NA) | (NA:NA) | ( ) | 0.2 | −0.37 | 0.6322 |
| | CT | | | | | | | | | |
| 151 | TGTCTTGCAGGCCGTCATGC | 0.02612 | 0.01998 | hsa-mir-431- | (0:−1) | (:) | ( ) | 0.2 | 0.613 | −0.6042 |
| | | | | 5p | | | | | | |
| 152 | CGAATCATTATTTGCTGCT | 0.02532 | 0.02532 | hsa-mir- | (0:−3) | (:) | ( ) | 0.1 | 1.521 | −0.0129 |
| | | | | 15b-3p | | | | | | |
| 153 | CAGCAGCAATTCATGTTTTG | 0.02138 | 0.02138 | hsa-mir-424- | (0:0) | (:A) | ( ) | 0.1 | 0.241 | −0.3669 |
| | AAA | | | 5p | | | | | | |
| 154 | ACCAATATTACTGTGCTGCT | 0.0205 | 0.01422 | hsa-mir-16- | (−1:−3) | (:) | ( ) | 0.2 | 3.128 | 1.1757 |
| | | | | 2-3p | | | | | | |
| 155 | TTCAAGTAATCCAGGATAG | −0.02004 | 0.02004 | hsa-mir-26a- | (0:2) | (:) | (22: G > T) | 0.1 | 3.007 | 4.1471 |
| | GCTTT | | | 5p | | | | | | |
| 156 | TTGAGAACTGAATTCCATG | 0.01968 | 0.01968 | hsa-mir- | (−1:−1) | (:) | ( ) | 0.1 | 1.968 | 0.5389 |
| | GGT | | | 146a-5p | | | | | | |
| 157 | TATTGCACATTACTAAGTTG | 0.01865 | 0.01865 | hsa-mir-32- | (0:−2) | (:) | ( ) | 0.1 | 3.749 | 1.603 |
| | | | | 5p | | | | | | |
| 158 | TGACCTATGAATTGACAGC | −0.01793 | 0.01793 | hsa-mir-215- | (1:2) | (:) | (18: A > C, | 0.1 | −0.659 | 0.189 |
| | CTA | | | 5p | | | 20: A > T) | | | |
| 159 | ACTGTAAACGCTTTCTGATG | −0.01783 | 0.01783 | hsa-mir- | (0:0) | (:) | ( ) | 0.1 | 1.014 | 1.2253 |
| | | | | 3607-3p | | | | | | |
| 200 | CATTGCACTTGTCTCGGTCT | −0.01738 | 0.01738 | hsa-mir-25- | (0:0) | (:AT) | ( ) | 0.1 | 0.719 | 1.4522 |
| | GAAT | | | 3p | | | | | | |
| 201 | ATAAAGTAGAAAGCACTAC | 0.01695 | 0.01695 | hsa-mir-142- | (1:0) | (:) | ( ) | 0.1 | 2.536 | 0.3764 |
| | T | | | 5p | | | | | | |
| 202 | AAGTGCAATGATGAAAGGG | 0.01537 | 0.01537 | hsa-mir- | (1:−1) | (A:) | (9: T > G,1 | 0.1 | 0.631 | −0.6633 |
| | CA | | | 130a-3p | | | 1: A > T) | | | |
| 203 | ACCATAAAGTAGAAAGCAC | 0.01523 | 0.01523 | hsa-mir-142- | (−1:−2) | (A:) | ( ) | 0.1 | 1.096 | −0.3697 |
| | TA | | | 5p | | | | | | |
| 204 | CCCCACTGCTAAATTTGACT | −0.01424 | 0.01424 | <NA> | (NA:NA) | (NA:NA) | ( ) | 0.1 | −0.076 | 1.0335 |
| | GGCTTT | | | | | | | | | |
| 205 | TGTCAGTTTGTCAAATACCC | 0.01423 | 0.01423 | hsa-mir-223- | (0:2) | (:A) | ( ) | 0.1 | 0.507 | −0.6124 |
| | CAAGA | | | 3p | | | | | | |
| 206 | TACCCAGTAGAACCGAATTT | −0.01326 | 0.01326 | hsa-mir- | (0:−1) | (:) | (5: T > A) | 0.1 | −0.197 | 0.5859 |
| | GT | | | 10b-5p | | | | | | |
| 207 | TTTGTTCGTTCGGCTCGCGT | −0.01282 | 0.01282 | hsa-mir-375 | (0:0) | (:) | (20: G > A) | 0.1 | −0.245 | 1.5709 |
| | AA | | | | | | | | | |
| 208 | ATGCTGCCAGTTGAAGAAC | 0.01218 | 0.01218 | hsa-mir-22- | (0:0) | (:A) | (1: A > T) | 0.1 | 0.462 | −0.555 |
| | TGTA | | | 3p | | | | | | |
| 209 | TGAGAACCACGTCTGCTCT | 0.01124 | 0.01124 | hsa-mir-589- | (0:−2) | (:) | ( ) | 0.1 | 0.523 | −0.2778 |
| | G | | | 5p | | | | | | |
| 210 | CTGCCAATTCCATAGGTCAC | −0.0098 | 0.0098 | hsa-mir-192- | (0:0) | (:T) | ( ) | 0.1 | 0.349 | 1.5762 |
| | AGT | | | 3p | | | | | | |
| 211 | TAGCTTATCAGACTGATGTT | 0.00974 | 0.00974 | hsa-mir-21- | (0:0) | (:GA) | ( ) | 0.1 | 0.626 | 0.2759 |
| | GAGA | | | 5p | | | | | | |
| 212 | GTAGCTTATCAGACTGATGT | 0.00953 | 0.00953 | hsa-mir-21- | (−1:2) | (:) | ( ) | 0.1 | 1.628 | 0.0433 |
| | TGACT | | | 5p | | | | | | |
| 213 | TTTGGTCCCCTTCAACCAGC | −0.00945 | 0.00945 | hsa-mir- | (0:0) | (:A) | ( ) | 0.1 | −0.62 | −0.0075 |
| | TGA | | | 133a-3p | | | | | | |
| 214 | TGTAATAGCAACTCCATGTG | −0.00844 | 0.00844 | hsa-mir-194- | (0:1) | (:) | (5: C > T) | 0.1 | −0.638 | 0.24 |
| | GAA | | | 5p | | | | | | |
| 215 | GGGACCTATGAATTGACAG | 0.00774 | 0.00774 | hsa-mir-192- | (2:0) | (GG:) | (17: C > A) | 0.1 | 0.989 | −0.4886 |
| | AC | | | 5p | | | | | | |
| 216 | TAAGGTGCATCTAGTGCAG | 0.00772 | 0.00772 | hsa-mir- | (0:−1) | (:) | (19: T > A) | 0.1 | 2.295 | 0.6414 |
| | ATA | | | 18b-5p | | | | | | |
| 217 | GTACTGGAAAGTGCACTTG | −0.00721 | 0.00721 | <NA> | (NA:NA) | (NA:NA) | ( ) | 0.1 | −0.395 | 1.7345 |
| | GACGAACA | | | | | | | | | |
| 218 | CCCGGGGCTACGCCTGTCT | −0.00713 | 0.00713 | <NA> | (NA:NA) | (NA:NA) | ( ) | 0.1 | 1.856 | 2.7329 |
| | GAGCGTCGCT | | | | | | | | | |
| 219 | AAAGCTGGGTTGAGAGGG | −0.00655 | 0.00655 | hsa-mir- | (1:2) | (:) | ( ) | 0.1 | 0.172 | 0.9853 |
| | CGAAA | | | 320a | | | | | | |
| 220 | CATAAAGTAGAAAGCACTA | 0.00604 | 0.00537 | hsa-mir-142- | (0:−2) | (:) | ( ) | 0.2 | 2.95 | 1.1211 |
| | | | | 5p | | | | | | |
| 221 | TGTCAGTTTGTCAAATAC | 0.00602 | 0.00602 | hsa-mir-223- | (0:−4) | (:) | ( ) | 0.1 | 2.716 | −0.261 |
| | | | | 3p | | | | | | |
| 222 | TCCGGTGAGCTCTCGCTGG | 0.00578 | 0.00578 | hsa-mir- | (−1:1) | (T:) | (9: G > C) | 0.1 | 0.207 | −0.4932 |
| | CC | | | 4792 | | | | | | |
| 223 | TATAAAGTAGAAAGCACTA | 0.00555 | 0.00555 | hsa-mir-142- | (1:−1) | (T:) | ( ) | 0.1 | 0.13 | −0.6931 |
| | C | | | 5p | | | | | | |
| 224 | TGCTGCCAGTTGAAGAACT | 0.00546 | 0.00546 | hsa-mir-22- | (2:0) | (T:) | ( ) | 0.1 | 0.158 | −0.6517 |
| | GT | | | 3p | | | | | | |
| 225 | AGCTCGGTCTGAGGCCCCT | −0.00518 | 0.00518 | hsa-mir-423- | (0:2) | (:) | (23: C > T) | 0.1 | 0.091 | 1.3932 |
| | CAGTTT | | | 3p | | | | | | |
| 226 | TGTCAGTTTGTCAAATACCC | 0.00464 | 0.00464 | hsa-mir-223- | (0:2) | (:) | (22: A > T) | 0.1 | 0.204 | −0.642 |
| | CATG | | | 3p | | | | | | |
| 227 | ATCACAGTGGCTAAGTTCC | 0.00413 | 0.00413 | hsa-mir-27a- | (1:−2) | (A:) | ( ) | 0.1 | 0.487 | −0.6176 |
| | | | | 3p | | | | | | |
| 228 | TGAGAACTGAATTCCATAG | 0.0039 | 0.0039 | hsa-mir- | (0:−1) | (:AA) | ( ) | 0.1 | 1.542 | 0.5058 |
| | GCAA | | | 146b-5p | | | | | | |
| 229 | TGGGTCTTTGCGGGCGAGA | −0.00383 | 0.00383 | hsa-mir- | (0:0) | (:) | ( ) | 0.1 | 1.582 | 2.1855 |
| | TGA | | | 193a-5p | | | | | | |
| 230 | TACCCTGTAGAACCGGATTT | −0.00313 | 0.00313 | hsa-mir- | (0:−2) | (:) | (15: A > G) | 0.1 | −0.657 | −0.2565 |
| | G | | | 10b-5p | | | | | | |
| 231 | TGAGGGAGTAGATTGTATA | 0.00301 | 0.00301 | hsa-let-7a- | (0:−1) | (:) | (5: T > G,1 | 0.1 | 1.916 | 0.1598 |
| | GT | | | 5p | | | 1: G > A) | | | |
| 232 | TACCCTGTTGAACCGAATTT | −0.00297 | 0.00297 | hsa-mir- | (0:−1) | (:) | (8: A > T) | 0.1 | −0.159 | 0.3187 |
| | GT | | | 10b-5p | | | | | | |
| 233 | TAAGGTGCATCTAGTGCAG | 0.00245 | 0.00245 | hsa-mir-18a- | (0:−2) | (:) | ( ) | 0.1 | 2.559 | 0.72 |
| | AT | | | 5p | | | | | | |
| 234 | GAGAACTGAATTCCATAGG | 0.0021 | 0.0021 | hsa-mir- | (1:2) | (:) | 0 | 0.1 | 0.549 | −0.328 |
| | CTGT | | | 146b-5p | | | | | | |
| 235 | TAGCAGCACGCAAATATTG | 0.00209 | 0.00209 | hsa-mir-16- | (0:0) | (:) | (10: T > C) | 0.1 | 0.28 | −0.5687 |
| | GCG | | | 5p | | | | | | |
| 236 | GGCTCGTTGGTCTAGGGG | −0.0019 | 0.0019 | hsa-mir- | (0:−2) | (:) | (5: C > G) | 0.1 | −0.534 | 0.0195 |
| | | | | 4448 | | | | | | |
| 237 | CAGCAGCAATTCATGTTTTG | 0.00173 | 0.00173 | hsa-mir-424- | (0:−2) | (:) | ( ) | 0.1 | 0.987 | −0.0245 |
| | | | | 5p | | | | | | |
| 238 | AACATTCAACGCTGTCGGT | −0.00169 | 0.00169 | hsa-mir- | (0:−3) | (:) | (8: T > A, | 0.1 | 3.67 | 3.8391 |
| | G | | | 181b-5p | | | 9: T > C) | | | |
| 239 | ATGCAGCACGTAAATATTG | 0.00169 | 0.00169 | hsa-mir-16- | (2:0) | (AT:) | ( ) | 0.1 | 0.338 | −0.6428 |
| | GCG | | | 5p | | | | | | |
| 240 | TGCCGACGGGCGCTGACCC | −0.00159 | 0.00159 | <NA> | (NA:NA) | (NA:NA) | ( ) | 0.1 | −0.369 | 0.6898 |
| | CCTT | | | | | | | | | |
| 241 | ATTGGTCGTGGTTGTAGTC | −0.00106 | 0.00106 | <NA> | (NA:NA) | (NA:NA) | ( ) | 0.1 | −0.405 | 0.4592 |
| | CGTGCGAGAA | | | | | | | | | |
| 242 | TGGCAGTGTCTTAGCTGGT | 0.001 | 0.001 | hsa-mir-34a- | (0:−1) | (:) | ( ) | 0.1 | 1.828 | 0.6251 |
| | TG | | | 5p | | | | | | |
| 243 | TGTCAGTTTGTCAAATA | 0.00095 | 0.00095 | hsa-mir-223- | (0:−5) | (:) | ( ) | 0.1 | 0.047 | −0.6931 |
| | | | | 3p | | | | | | |
| 244 | ACCCTGAGACCCTAACTTGT | 0.00016 | 0.00016 | hsa-mir- | (1:0) | (A:) | ( ) | 0.1 | 0.322 | −0.5771 |
| | GA | | | 125b-5p | | | | | | |
| 245 | TGGCAGTTTGTCAAATACC | 0.00011 | 0.00011 | hsa-mir-223- | (0:−3) | (:) | (2: T > G) | 0.1 | 1.467 | −0.5979 |
| | | | | 3p | | | | | | |

TABLE 5
Identified sRNA biomarkers in colon epithelium tissue that are associated with Diverticular disease.

| SEQ ID | | | | | | | | | | |
|---|---|---|---|---|---|---|---|---|---|---|
| NO: | Marker | importance | imp_SE | sRNA_name | ref | ext | swaps | chosen | thislbl | otherlbl |
| 246 | ACTGGACTTGGAGTCAGAAGGCA | 1.3057 | 0.12197 | hsa-mir- | (0:0) | (:ATAT) | (9: A > G, | 1 | 1.458 | −0.67 |
| | TAT | | | 422a | | | 11: G > A) | | | |
| 247 | TCGACCGGACCTCGACCGGCTAG | 0.23143 | 0.11311 | hsa-mir- | (0:2) | (:A) | (21: C > A) | 0.4 | 1.008 | −0.59 |
| | A | | | 1307-5p | | | | | | |
| 248 | TCAGCACCAGGATATTGTTGGA | 0.11606 | 0.05936 | hsa-mir- | (0:−1) | (:) | ( ) | 0.4 | 1.535 | −0.58 |
| | | | | 3065-3p | | | | | | |
| 249 | TGTAACCGCAACTCCATGTGGA | 0.09378 | 0.05427 | hsa-mir- | (0:0) | (:) | (6: A > C) | 0.3 | 1.788 | −0.39 |
| | | | | 194-5p | | | | | | |
| 250 | ACTGGACTTGGAGTCAGAAGGCA | 0.08715 | 0.04571 | hsa-mir- | (0:0) | (:ATTA) | (9: A > G, | 0.3 | 1.098 | −0.67 |
| | TTA | | | 422a | | | 11: G > A) | | | |
| 251 | AACACTGTCTGGTAAAGATGGC | 0.08212 | 0.0662 | hsa-mir- | (1:1) | (:) | ( ) | 0.2 | 1.265 | −0.63 |
| | | | | 141-3p | | | | | | |
| 252 | TGTAAACATCCTACACTCTCAGCT | 0.08206 | 0.03761 | hsa-mir- | (0:1) | (:TA) | ( ) | 0.5 | 0.138 | −0.69 |
| | TA | | | 30c-5p | | | | | | |
| 253 | ACTGGACTTTGAGTCAGAAGGCA | 0.06028 | 0.04522 | hsa-mir- | (0:0) | (:A) | (9: A > T, | 0.3 | 0.671 | −0.65 |
| | | | | 422a | | | 11: G > A) | | | |
| 254 | ACTGGACTTGGAGCCAGAAGGCA | 0.05242 | 0.04482 | hsa-mir- | (0:2) | (:AA) | (20: T > G) | 0.2 | 0.921 | −0.65 |
| | A | | | 378f | | | | | | |
| 255 | GTAACAGCAACTCCATGTGGAAA | 0.04186 | 0.02857 | hsa-mir- | (1:1) | (:A) | ( ) | 0.2 | 0.92 | −0.67 |
| | | | | 194-5p | | | | | | |
| 256 | ACTGGACTTGGAGTCAGAAGGCA | 0.03645 | 0.01948 | hsa-mir- | (0:0) | (:AATA) | (9: A > G, | 0.5 | −0.038 | −0.69 |
| | ATA | | | 422a | | | 11: G > A) | | | |
| 257 | CTGGACTTGGAGTCAGAAGGCAG | 0.0346 | 0.02916 | hsa-mir- | (1:2) | (:AGA) | (12: C > T, | 0.2 | 0.159 | −0.68 |
| | A | | | 378f | | | 19: T > G) | | | |
| 258 | TGATATGTTTGATATATTAGGTTA | 0.03153 | 0.02537 | hsa-mir- | (0:1) | (:A) | ( ) | 0.2 | 1.842 | −0.53 |
| | | | | 190a-5p | | | | | | |
| 259 | TGAAATGTTTAGGACCACTAGAA | 0.02779 | 0.02185 | hsa-mir- | (1:1) | (:AT) | ( ) | 0.2 | 0.309 | −0.68 |
| | T | | | 203a-3p | | | | | | |
| 300 | TGGACTTGGAGTCAGAAGGCAT | 0.02407 | 0.01645 | hsa-mir- | (2:0) | (:AT) | ( ) | 0.2 | 0.622 | −0.66 |
| | | | | 378a-3p | | | | | | |
| 301 | TGTAACAGCAACTCCATGTGGAC | 0.01862 | 0.01862 | hsa-mir- | (0:2) | (:A) | ( ) | 0.1 | 0.327 | −0.58 |
| | TA | | | 194-5p | | | | | | |
| 302 | TCGACCGGACCTCGACCGGCTA | 0.01749 | 0.01519 | hsa-mir- | (0:0) | (:A) | ( ) | 0.2 | 1.518 | −0.6 |
| | | | | 1307-5p | | | | | | |
| 303 | TGAGATGAAGCACTGTAGCTCAT | 0.01455 | 0.01455 | hsa-mir- | (0:1) | (:TA) | ( ) | 0.1 | 0.975 | −0.61 |
| | A | | | 143-3p | | | | | | |
| 304 | TTTCAGTCGGATGTTTGCAGCAA | 0.01444 | 0.01444 | hsa-mir- | (1:0) | (:AA) | (16: A > G) | 0.1 | 0.141 | −0.69 |
| | | | | 30e-3p | | | | | | |
| 305 | GACCTATGAATTGACAGCCAT | 0.01188 | 0.00963 | hsa-mir- | (2:1) | (:T) | (17: A > C) | 0.2 | 1.014 | −0.58 |
| | | | | 215-5p | | | | | | |
| 306 | CCACTGCCCCAGGTGCTGCTGGA | 0.01092 | 0.01092 | hsa-mir- | (−2:0) | (:A) | ( ) | 0.1 | 0.692 | −0.6 |
| | | | | 324-3p | | | | | | |
| 307 | CTGACCTATGAATTGACAGCCAT | 0.0102 | 0.0102 | hsa-mir- | (0:1) | (:TGA) | ( ) | 0.1 | 0.583 | −0.63 |
| | GA | | | 192-5p | | | | | | |
| 308 | ACCACAGGGTAGAACCACGGACG | 0.00927 | 0.00927 | hsa-mir- | (1:2) | (:GA) | ( ) | 0.1 | 0.682 | −0.58 |
| | A | | | 140-3p | | | | | | |
| 309 | TCGACCGGACCTCGACCGGCTGA | 0.00896 | 0.00896 | hsa-mir- | (0:0) | (:GA) | ( ) | 0.1 | −0.463 | −0.68 |
| | | | | 1307-5p | | | | | | |
| 310 | TGGCTCAGTTCAGCAGGAACAGG | 0.00641 | 0.00641 | hsa-mir-24- | (0:2) | (:) | ( ) | 0.1 | 0.543 | −0.6 |
| | A | | | 3p | | | | | | |
| 311 | AGCTTATCAGACTGATGTTGAAA | 0.00487 | 0.00487 | hsa-mir-21- | (1:0) | (:AA) | ( ) | 0.1 | 0.052 | −0.66 |
| | | | | 5p | | | | | | |
| 312 | ATCACATTGCCAGGGATAAAA | 0.00469 | 0.00469 | hsa-mir-23c | (0:−3) | (:AA) | (13: T > G, | 0.1 | 0.333 | −0.66 |
| | | | | | | | 17: T > A) | | | |
| 313 | TCAACAAAATCACTGATGCTGGA | 0.0018 | 0.0018 | hsa-mir- | (0:0) | (:) | ( ) | 0.1 | 0.71 | −0.53 |
| | | | | 3065-5p | | | | | | |
| 314 | ACATTGCCAGGGATTTCCA | 0.00084 | 0.00084 | hsa-mir- | (3:1) | (:) | ( ) | 0.1 | 1.31 | −0.57 |
| | | | | 23a-3p | | | | | | |
| 315 | AACACTGTCTGGTAAAGATG | 0.00065 | 0.00065 | hsa-mir- | (1:−1) | (:) | ( ) | 0.1 | −0.094 | −0.69 |
| | | | | 141-3p | | | | | | |

### Multi-Class Disease Classification

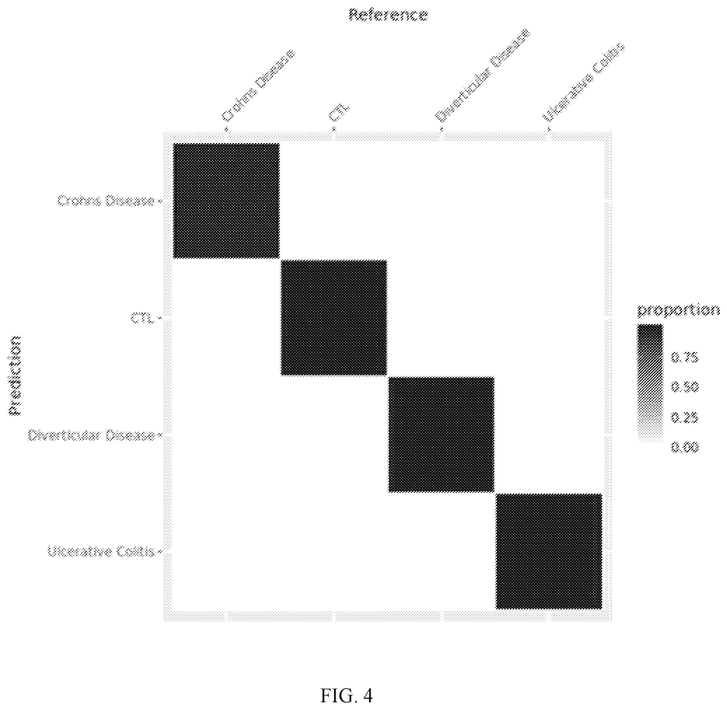

The disease classifier was trained based on the positive or negative markers of the sRNA panels, as well as the presence or absence of the sRNAs in the panels identified above for Controls, Crohn's disease, ulcerative colitis, and diverticular disease. In order to assess the accuracy of the computational model when the class metrics were all combined, a test was run to evaluate the model's identification predictive power against reference samples of each class. It was found that the model had an accuracy rate of 98%. FIG. 4 depicts a heat map showing the proportion of accurate predictions of disease class against their true reference identities. These results are also shown in the matrix below:

| | Reference | | | |
|---|---|---|---|---|
| | Crohn’s | | Diverticular | Ulcerative |
| Prediction | Disease | Control | Disease | Colitis |
| Crohn’s | 116 | 0 | 0 | 0 |
| Disease | | | | |
| Control | 0 | 179 | 0 | 0 |
| Diverticular | 0 | 0 | 59 | 4 |
| Disease | | | | |
| Ulcerative | 4 | 1 | 1 | 226 |
| Colitis | | | | |

### Example 2: Use of Spike-In Data

This example illustrates a use of spike-in data obtained from an entire sequencing run using sRNA extracted from 137, 0.5mL cerebrospinal fluid samples using the miRNeasy Serum/Plasma Advanced Kit (Qiagen).

An RNA spike-in mix was used comprising five calibrators which were pooled, and the pool was spiked into each sample before library preparation, such that the final concentration of each spike in the sample was as follows:

Calibrator 1=0.0001 amol/μL

Calibrator 2=0.001 amol/μL

Calibrator 3=0.01 amol/μL

Calibrator 4=0.1 amol/μL

Calibrator 5=1.0 amol/μL

The samples (including the spike-in mixture) were subjected to library preparation including 3′ and 5′ adaptor ligation, followed by reverse transcription and then PCR amplification to add unique barcodes to each sample using the NextFlex Small RNA Library Preparation Kit v3.0 (BIOO) on a Sciclone iQ NGS Workstation (PerkinElmer).

The samples were pooled to a final concentration of 0.65nM and were sequenced on a NovaSeq 6000 Sequencing System (Illumina) using an S2 flow cell run at 101 bp per direction. Using this schema, each sample was sequenced at a depth of 12,000,000 reads or more. The data was trimmed using a trimming algorithm.

Spike-ins were mapped using a spike-in reference library. The reads were converted to TRPM (trimmed reads per million reads). The data was plotted and R-squared was calculated. FIG. 5 illustrates the result of plotting the data for the entire run 137 samples (R₂=0.989).

### Example 3: Subtyping of Idiopathic Pulmonary Fibrosis

Idiopathic pulmonary fibrosis (IPF) is an irreversible, fatal disease. Incidence rates of IPF vary between 2.5-16.0 per 100,000 people in the US, Europe and Asia. Based on these incidence rates, it can be estimated that over 1 million world-wide are battling this disease each year. IPF presents symptomatically with dyspnea, cough and decreased lung function over time. Diagnosis of IPF is a complex procedure that often takes over a year and requires a multi-disciplinary team comprised of pulmonologists, thoracic radiologists and pathologists who perform clinical tests, bronchoscopies, lung biopsies and histology.

IPF patients have a poor prognosis with >50% having mortality in less than 5 years from the time of diagnosis. Pathology of IPF lung tissue shows distortion of lung architecture due to uncontrolled proliferation of fibroblasts and excessive deposition of extracellular matrix molecules. However, overall survival is not absolute, and patients have variable trajectories ranging from slow progressive disease in some patients and rapid deterioration in others. Therefore, heterogeneity may be linked to genetic and environmental factors impacting disease drivers and other genes required for disease maintenance that are poorly understood.

To identify biomarkers that could predict outcomes in IPF patients and better understand the drivers of disease, it was hypothesized that blood-based small RNA (sRNA) biomarkers could be discovered using the machine learning discovery platform described herein. To test this hypothesis, IPF samples from the observational, multi-site, prospective longitudinal PROFILE study were evaluated according to embodiments of the invention. The PROFILE study analyzed the statistical correlation of 123 serum proteins. See, Maher T M, et al., PROFILEing idiopathic pulmonary fibrosis: rethinking biomarker discovery. European Respiratory Review 22, 148-152 (2013); Maher, T M, et al., An epithelial biomarker signature for idiopathic pulmonary fibrosis: an analysis from the multicenter PROFILE cohort study. The Lancet Respiratory Medicine 5, 946-955 (2013). The aims of the present research were to further classify (i.e., subtype) IPF. Results demonstrate that an sRNA-signature (based on a panel of 86 sRNAs) can classify IPF from Control samples with 100% accuracy, and can type IPF samples into several distinct clusters. 50 IPF disease samples and 170 healthy donor samples were used (PAXgene biospecimens). In particular, 170 age and sex matched controls with corresponding diffusing capacity of the lungs for carbon monoxide (DLco), forced expiratory volume, first breath (Fev1), forced vital capacity (Fvc), and Fev1:Fvc Ratio metadata were selected.

Blood RNA was extracted using the PAXgene Blood RNA Extraction Kit (QIAGEN) on a QIACube Connect (QIAGEN) automated liquid handler. RNA quantities were assessed using the RNA HS Assay Kit (Thermo) on a Qubit 4 Fluorometer (Thermo). RNA Integrity Scores (RIN) were assessed using the LabChip RNA HS Assay Kit (PerkinElmer) on a LabChip GX Touch (PerkinElmer). 250 ug of total RNA from each sample was aliquoted into a 96-well plate. A cocktail of spike-in calibrators was added to each sample to monitor quality control and facilitate downstream normalization during analytics. Next generation sequencing (NGS) libraries were prepared using the NextFlex Small RNA Library Prep Kit v3 (BIOO) on a Sciclone iQ NGS Workstation (PerkinElmer) which incorporates unique i7/i5 dual indexes to each sample to support multiplexed sequencing. Libraries were quantified using the lx dsDNA HS Assay Kit (Thermo) on a Qubit 4 Fluorometer (Thermo). Library fragmentation analysis was assessed using the LabChip DNA 3K NGS Assay Kit (PerkinElmer). Libraries were pooled at a concentration of 1.0 nM. Pooled libraries were sequenced at a target depth of 40 million paired end reads per sample using an S2 Flow Cell Kit (Illumina) on a NovaSeq 6000 Sequencing System (Illumina).

Small RNA sequencing data quality was assessed using FASTQC. Reads passing filter (Q-score >00%) were processed and annotated using a suite of trimming and short read alignment algorithms designed to annotate small RNAs. This short read alignment approach permits annotation of templated and non-templated nucleotide additions at the 5′ and 3′ end of small RNAs to provide information on gene targeting and cellular localization to exosomes. This short read alignment approach also enables mapping of over 10,000 times more unique small RNA genes compared to annotated libraries of microRNA. Analysis showed a consistent profile across mapped reads between 17 - 43 base pairs in length that were used for analysis.

IPF samples and CTL samples were each randomly divided into training and test sets in a 90:10 ratio (training:test) for use in Monte Carlo, cross-validation runs. Following the Monte Carlo runs, the data were analyzed using a suite of artificial intelligence algorithms leveraging supervised and unsupervised machine learning (ML) to identify predictive sRNA-signatures. The ML algorithms created a model using the set of training samples and then measured the accuracy using the set of test samples.

Specifically, sRNAs with a minimum class frequency of >5% in the training samples were selected. An elastic net algorithm was used to reduce the panel using hyper-features such as sRNA gene families and 3′ non-templated nucleotide additions. The test samples were analyzed using a Support Vector Machine (SVM) and then Receiving Operator Characteristics (ROC) were used to measure the Area Under the Curve, Accuracy, Sensitivity, Specificity, Positive Predictive Value, Negative Predictive Value and F1-Scores.

In 96 Monte Carlo cross-validation runs, an sRNA-signature of 86 small RNA genes was identified, which provided 99.3% accuracy (95% CI 98.5-100%, p<0.00001) in discriminating IPF samples from CTL samples. The disease prediction model also produced a SVM score for each sample, between 0.0 and 1.0. Scores over 0.5 were classified as diseased. Approximately 94% of the Disease Probability Scores of CTL samples were between 0.0-0.1. The tight grouping of samples indicated that the CTL samples are a homogenous group. In contrast, the IPF samples showed a distribution that was spread over a wide, flat area with several distinct peaks suggesting heterogeneity.

There were 86 small RNA genes in the sRNA-signature that discriminated IPF from CTL samples. In this signature, 37 (43%) sRNAs were up-regulated and 49 (57%) were down-regulated in IPF samples compared to CTL samples. The signature was comprised of 71 miRNA isoforms, 9 intergenic derived sRNAs that map to introns and exons of protein coding genes, 3 rRNA-derived sRNAs, 2 piRNA isoforms, and 1 yRNA-derived sRNA. There were: 4 miRNA isoforms with >10-fold over-expression in IPF samples compared to CTL; 7 miRNA isoforms and 3 intergenic sRNAs with <10-fold down-regulation in IPF samples compared to CTL.

For unsupervised hierarchical clustering, Euclidean distance was calculated using the 86 sRNA genes from the predictive sRNA-signature. Samples were grouped using complete-linkage agglomerative clustering. Results revealed three IPF subtypes and indicated that the 86 predictive small RNA genes were not evenly distributed and expressed in all of the IPF samples.

Principle component (PC) analysis showed separation of the IPF samples using the subtypes groups assigned from the unsupervised hierarchical clustering analysis. Unit variance scaling was applied; Singular Value Decomposition (SVD) with imputation was used to calculate principle components. Samples were plotted using PC1 (29%) and PC2 (19%). Prediction ellipses show the 0.95 probability that a new observation from the same group will fall inside the ellipse.

Target prediction algorithms were used to identify targets for the 86 small RNA genes in the sRNA-signature. The target prediction process started by analyzing each of the 86 small RNA genes from the sRNA-signature that classified IPF from CTL with 99.3% accuracy and stratified the IPF samples into subgroups. Within these 86 genes, 40 unique ‘seeds’ were found. Using these 40 seeds, the target prediction algorithm resulted in 14,280 predicted genes with a p<0.01 and an FDR<0.05. Three cross-validation reference searches were used to weight predictions. Biological directionality was applied to parse out functionally relevant targets. Gene Ontology Term Enrichment for ‘cellular component’ was used to parse small RNA genes and targets.

The results of this study identified an sRNA-signature that was able to discriminate IPF samples from CTL samples with 99.3% accuracy and was also able to stratify IPF samples into three principal subtypes. The sRNA-signature comprises a panel of 86 small RNA genes. Analyzing the biological significance of the sRNA-signature predicted dysregulation of several biological pathways.

### Example 4: Reduction of Candidate sRNAs

Small RNA sequencing data derived from PAXgene Blood RNA of 511 patients diagnosed with Idiopathic Pulmonary Fibrosis (IPF) and 221 normal, healthy control (CTL) subjects was analyzed using machine learning to identify biomarkers capable of classifying IPF or CTL. Three different classification runs were tested allowing the classifier to select: (1) all small RNA features, (2) only small RNAs that map perfectly to the human genome and disallow intergenic mapping small RNAs, and (3) only microRNA isoforms, transfer RNA-derived fragments, ribosomal RNA-derived fragments without swaps.

In each case a model was trained on 49 IPF and 182 CTL samples, and tested on 462 and 39 CTL samples. In each case the classifier was allowed to select up to 3,000 small RNA features per class with a minimum training set frequency of 10%. In each case the elastic net reduced the final biomarker panel to a maximum number of 96 small RNAs per model.

Results show that restriction of the pre-selection filter to only allow microRNA isoforms, transfer RNA-derived fragments, ribosomal RNA-derived fragments without swaps gave the best performance with an AUC=71.2 and Accuracy=92.6%. Allowing all small RNAs into the pre-selection filter gave an AUC=66.7% and Accuracy=18.3%. Restricting the pre-selection filter to only allow small RNAs that map perfectly to the human genome and disallow intergenic mapping small RNAs gave an AUC=69.3 and Accuracy=45.8%.

In addition, a pre-selection can employ information concerning miRNA seed sequence. Small RNA sequencing data was aggregated from 4 studies (GSE110907, GSE62182, GSE83527 and TCGA-LUAD) containing a total of 693 cancerous (LUAD) and 231 normal adjacent tissue (CTL) lung biopsy samples. These samples were analyzed using machine learning with cross-validation designed to classify LUAD or CTL tissue.

In an exemplary investigation the system was trained on 645 LUAD and CTL samples from GSE62182, GSE83527 and TCGA-LUAD and tested on 48 LUAD and CTL samples from GSE110907. In a second investigation, system was trained on 563 LUAD and 101 CTL samples from GSE110907 and TCGA-LUAD and tested on 130 LUAD and CTL samples from GSE2182 and GSE83527. In each case, 50 bootstrapped tests were run where the pre-selection algorithm was allowed to select either 2,000 or 6,000 sRNA features. Selected sRNAs were then aggregated based on matching seed sequence (nucleotides 2-8 from the 5′ end of the small RNA feature) or were left unaggregated. The seed aggregated or non-aggregated feature set was reduced using an elastic net algorithm that allowed a maximum of 96 small RNAs. The reduced feature set was used to train a support vector machine that tested samples from GSE110907 or GSE62182 and GSE83527.

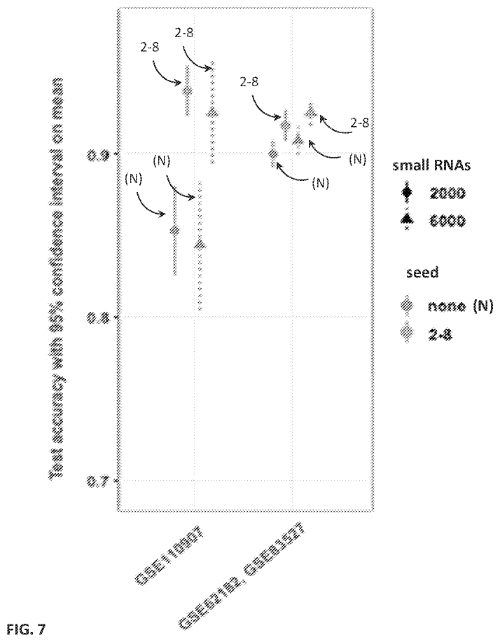

Results showed that pre-selection of 2,000 and 6,000 sRNAs gave comparable accuracy on tested samples. Whereas, support vector machines trained with values from seed aggregated feature sets gave enhanced classification performance when compared to the non-seed aggregated study. See FIG. 7.

| | Lung | |
|---|---|---|
| Study | Cancer | Control |
| GSE110907 | 48 | 49 |
| GSE62182 | 94 | 94 |
| GSE83527 | 36 | 36 |
| TCGA- | 515 | 52 |
| LUAD | | |
| Total | 693 | 231 |

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a nontransitory computer readable storage medium. For instance, the computer program product could contain the program modules shown and/or described in any combination of FIGS. 1 and 2. These program modules can be stored on a CD-ROM, DVD, magnetic disk storage product, USB key, or any other non-transitory computer readable data or program storage product.

1. A method for making a classifier for evaluating a subject for one or more biological conditions, comprising:
providing sRNA sequence data comprising the presence or absence, or abundance, of sRNA sequences across a set of discovery samples, the set of discovery samples representing the presence or absence of one or more biological conditions;
selecting candidate sRNA sequences whose presence or absence, or abundance, is correlative with the presence or absence of a biological condition; and
from the candidate sRNA sequences, training a classifier comprising features for evaluating a sample for the one or more biological conditions.

2. The method of claim 1, wherein the discovery samples are labelled as positive or negative for two or more biological conditions.

3. The method of claim 1, wherein the sRNA sequence data is processed by trimming 5′ and 3′ sequencing adaptors from sRNA sequence reads and without consolidating sRNA sequence variants based on a reference sequence or genetic locus.

4. The method of claim 3, wherein candidate sRNA sequences are selected based on the degree to which their presence or absence, or abundance, correlates to a biological condition.

5. The method of claim 4, wherein at least one candidate sRNA sequence is present in a plurality of discovery samples that are positive for a biological condition, and absent in all non-disease samples or all samples labeled with a different biological condition.

6. The method of claim 4, wherein candidate sRNA sequences are selected which individually predict, by their presence or abundance, for the presence or absence of a biological condition.

7. The method of claim 6, wherein candidate sRNA sequences are selected whose presence or abundance is predictive of the presence or absence of a biological condition, and with a p-value of at least 0.01.

8. The method of claim 7, wherein at least one candidate sRNA sequence is selected whose presence or abundance is predictive of the presence of absence of a biological condition, and with a p-value of at least 0.0001.

9. The method of claim 7, wherein at least one candidate sRNA sequence is selected whose presence or abundance is predictive of the presence or absence of a biological condition, and with a p-value of at least 0.000001.

10. The method of claim 7, wherein at least one candidate sRNA sequence is selected whose presence or abundance is predictive of the presence or absence of a biological condition, and with a p-value of at least 0.00000001.

11. The method of claim 7, wherein at least one candidate sRNA sequence is selected whose presence or abundance is predictive of the presence or absence of a biological condition, and with a p-value of at least 0.0000000001.

12. The method of claim 7, wherein candidate sRNA sequences are selected that are predictive, individually, for the presence or absence of at least two biological conditions.

13. The method of claim 1, wherein the set of discovery samples are sourced from at least two separate studies, and wherein the selected candidate sRNA sequences were each present in at least one sample from each study.

14. The method of claim 13, wherein the separate studies involve collection of biological samples at different sites.

15. The method of claim 14, wherein the separate studies further involve extraction of nucleic acid or sRNA at different sites.

16. The method of claim 15, wherein the separate studies further involve sRNA sequencing at different sites.

17. The method of any one of claims 1 to 16, wherein the set of discovery samples are further labeled for stage, grade, or severity of a biological condition, and where candidate sRNA sequences are selected whose read counts correlate with such stage, grade, or severity.

18. The method of claim 17, wherein the sRNA sequences were determined by sRNA sequencing using an endogenous sRNA control and/or a spike-in control, to normalize levels of sRNA sequences relative to the control(s).

19. The method of claim 18, wherein RNA from multiple samples are pooled for sequencing, with sequences from different samples containing an identifying sample tag sequence.

20. The method of claim 19, wherein candidate sRNA sequences have an average read count of at least 0.1 trimmed reads per million reads.

21. The method of claim 1, wherein candidate sRNA sequences are selected by identifying sRNA families having increased sequence diversity in a biological condition, and selecting sRNA sequences within the sRNA family as candidate sRNA sequences; and/or candidate sRNA sequences are selected that have sequence features associated with presence in exosomes.

22. The method of any one of claims 1 to 21, wherein the set of discovery samples represents the presence and absence of at least three biological conditions, or at least five biological conditions.

23. The method of claim 22, wherein the set of discovery samples represents the presence and absence of at least ten biological conditions.

24. The method of any one of claims 1 to 23, wherein the classifier is trained to classify samples based on the presence or absence, or abundance, of a panel of sRNA sequences, where the panel contains from about 4 to about 200 sRNA sequences per class, or from about 4 to about 100 sRNA sequences per class, or from about 4 to about 50 sRNA sequences per class.

25. The method of any one of claims 1 to 24, wherein the set of discovery samples comprise solid tissue samples, biological fluid samples, or cultured cells.

26. The method of claim 25, wherein the set of discovery samples are blood, serum, plasma, cerebrospinal fluid, urine, or saliva.

27. The method of claim 25, wherein the set of discovery samples are solid tissue biopsies.

28. The method of any one of claims 1 to 27, wherein the set of discovery samples includes at least 100 samples, including at least 10 samples that are positive for each of the at least two biological conditions.

29. The method of claim 28, wherein the discovery samples comprise at least 25 non-disease or healthy controls.

30. The method of any one of claims 1 to 29, wherein the classifier is trained using one or more of supervised, unsupervised, semi-supervised machine learning models such as, parametric/non-parametric distance measures, logistic regression, support vector machines, decision trees, random forests, neural networks, probit regression, Fisher's linear discriminant, Naive Bayes classifier, perceptron, quadratic classifiers, kernel estimation, k-nearest neighbor, learning vector quantization, and principal components analysis.

31. The method of claim 30, wherein the classifier is trained using linear support vector machine.

32. The method of claim 31, wherein sRNA sequence data from supplemental discovery samples is evaluated to reduce classifier features.

33. The method of any one of claims 1 to 32, wherein the biological conditions are conditions of the central nervous system.

34. The method of claim 33, wherein at least two biological conditions are neurodegenerative diseases involving symptoms of dementia.

35. The method of claim 33, wherein at least two biological conditions are selected from Alzheimer's Disease, Parkinson's Disease, Huntington's Disease, Mild Cognitive Impairment, Progressive Supranuclear Palsy, Frontotemporal Dementia, Lewy Body Dementia, and Vascular Dementia.

36. The method of claim 33, wherein at least two biological conditions are neurodegenerative diseases involving symptoms of loss of movement control.

37. The method of claim 36, wherein at least two biological conditions are selected from Alzheimer's Disease, Parkinson's Disease, Huntington's Disease, Multiple Sclerosis, Amyotrophic Lateral Sclerosis, and Spinal Muscular Atrophy.

38. The method of claim 33, wherein at least two biological conditions are demyelinating diseases, optionally including multiple sclerosis, optic neuritis, transverse myelitis, and neuromyelitis optica.

39. The method of any one of claims 1 to 32, wherein one or more biological conditions are selected from Alzheimer's Disease, Parkinson's Disease, Huntington's Disease, Multiple Sclerosis, Amyotrophic Lateral Sclerosis, and Spinal Muscular Atrophy; and training samples are labelled for disease stage, disease severity, drug responsiveness, or course of disease progression.

40. The method of any one of claims 1 to 32, wherein the biological conditions are cancers of different tissue or cell origin.

41. The method of claim 40, wherein the biological conditions include drug sensitive and drug resistant cancers.

42. The method of claim 40 or 41, wherein the biological sample from the subject is a tumor or cancer cell biopsy.

43. The method of any one of claims 1 to 32, wherein the biological conditions are inflammatory or immunological diseases, and optionally including one or more of Systemic Lupus Erythematosus (SLE), scleroderma, autoimmune vasculitis, diabetes mellitus (type 1 or type 2), Grave's disease, Addison's disease, Sjogren's syndrome, thyroiditis, rheumatoid arthritis, myasthenia gravis, multiple sclerosis, fibromyalgia, psoriasis, Crohn's disease, ulcerative colitis, diverticular disease, celiac disease, and a disease of organ fibrosis.

44. The method of claim 43, wherein the biological samples are blood, serum, or plasma.

45. The method of any one of claims 1 to 32, wherein the biological conditions are cardiovascular diseases optionally including stratification for risk of acute event.

46. The method of claim 45, wherein the cardiovascular diseases include one or more of coronary artery disease (CAD), myocardial infarction, stroke, congestive heart failure, hypertensive heart disease, cardiomyopathy, heart arrhythmia, congenital heart disease, valvular heart disease, carditis, aortic aneurysms, peripheral artery disease, and venous thrombosis.

47. The method of any one of claims 1 to 32, wherein at least two biological conditions are a disease subtype.

48. The method of claim 47, wherein the set of samples are not labeled for disease subtype of a complex disease, and a disease subtype classifier is trained using an unsupervised machine learning model; or the set of samples are only partially labeled for disease subtype of a complex disease, and a disease subtype classifier is trained using a semi-supervised machine learning model.

49. The method of claim 48, wherein sRNAs in the panel are mapped to target genes or pathways to identify druggable targets or therapeutic interventions for the disease subtypes.

50. A method for evaluating a subject for one or more biological conditions, comprising:
providing a biological sample of the subject, and determining the presence or absence, or the abundance, of sRNAs in an sRNA panel;
classifying the condition of the subject among one or more biological conditions using a disease classifier prepared according to any one of claims 1 to 49.

51. The method of claim 50, wherein the presence or absence, or abundance, of sRNAs in the sample is determined by quantitative PCR assays.

52. The method of claim 50, wherein the presence or absence, or abundance, of sRNAs in the sample is determined by sRNA sequencing, which optionally employs sRNA target capture.

53. The method of any one of claims 50 to 52, wherein the disease classifier classifies samples among at least three biological conditions, or at least five biological conditions.

54. The method of claim 53, wherein the disease classifier classifies among at least ten biological conditions.

55. The method of any one of claims 50 to 54, wherein the panel contains from about 4 to about 200 sRNAs, or from about 4 to about 100 sRNAs, or from about 4 to about 50 sRNAs.

56. The method of claim 55, wherein the biological sample comprises one or more of solid tissue samples, biological fluid samples, or cultured cells.

57. The method of claim 56, wherein the biological sample is blood, serum, plasma, cerebrospinal fluid, urine, or saliva.

58. The method of claim 56, wherein biological sample of the subject is a solid tissue biopsy.

59. The method of claim 57, wherein the classifier is trained using a discovery set representing biological conditions of the central nervous system.

60. The method of claim 59, wherein the subject exhibits symptoms consistent with a disease of the central nervous system.

61.l The method of claim 60, wherein the subject has symptoms of dementia.

62. The method of claim 60, wherein the subject has symptoms of loss of movement control.

63. The method of claim 61 or 62, wherein the subject is classified as having or not having one or more of Alzheimer's Disease, Parkinson's Disease, Huntington's Disease, Mild Cognitive Impairment, Progressive Supranuclear Palsy, Frontotemporal Dementia, Lewy Body Dementia, Vascular Dementia, Multiple Sclerosis, Amyotrophic Lateral Sclerosis, and Spinal Muscular Atrophy.

64. The method of claim 60, wherein the subject is classified as having or not having a demyelinating disease, optionally including one or more of multiple sclerosis, optic neuritis, transverse myelitis, and neuromyelitis optica.

65. The method of claim 60, wherein the subject is diagnosed or determined to have one or more of Alzheimer's Disease, Parkinson's Disease, Huntington's Disease, Multiple Sclerosis, Amyotrophic Lateral Sclerosis, and Spinal Muscular Atrophy; and the subject is classified for disease stage, disease severity, drug responsiveness, or course of disease progression.

66. The method of any one of claims 50 to 58, wherein the subject is at risk for cancer, is suspected of having a cancer, or is diagnosed as having cancer.

67. The method of claim 66, wherein the subject has cancer, and the sample is classified for one or more selected from drug sensitivity, drug resistance, and tissue origin.

68. The method of claim 67, wherein the biological sample from the subject is a tumor or cancer cell biopsy.

69. The method of any one of claims 50 to 58, wherein the subject presents with symptoms of an inflammatory or immunological disease.

70. The method of claim 69, wherein the subject's sample is classified for the presence or absence of one or more of Systemic Lupus Erythematosus (SLE), scleroderma, autoimmune vasculitis, diabetes mellitus (type 1 or type 2), Grave's disease, Addison's disease, Sjogren's syndrome, thyroiditis, rheumatoid arthritis, myasthenia gravis, multiple sclerosis, fibromyalgia, psoriasis, idiopathic pulmonary fibrosis, Crohn's disease, ulcerative colitis, diverticular disease and celiac disease.

71. The method of claim 69 or 70, wherein the biological samples are blood, serum, or plasma.

72. The method of any one of claims 50 to 58, wherein the disease conditions are cardiovascular diseases optionally including stratification for risk of acute event.

73. The method of claim 72, wherein the cardiovascular diseases include one or more of coronary artery disease (CAD), myocardial infarction, stroke, congestive heart failure, hypertensive heart disease, cardiomyopathy, heart arrhythmia, congenital heart disease, valvular heart disease, carditis, aortic aneurysms, peripheral artery disease, and venous thrombosis.

74. The method of any one of claims 50 to 73, wherein the subject is classified for a disease subtype of a complex disease.
